(19) United States
(12) Patent Application Publication
(10) Pub. No.: US 2025/0297177 A1
(43) Pub. Date: Sep. 25, 2025

(54) PROCESS TO UPGRADE AROMATIC WASTE STREAMS

(71) Applicant: Basell Poliolefine Italia S.r.l., Milan (IT)

(72) Inventors: David S. Mannel, Humble, TX (US); Sandor Nagy, Seabrook, TX (US); Barbara Kimmich, Houston, TX (US); Karl P. Rufener, Humble, TX (US); Daniel F. White, Houston, TX (US); Robert J. Casey, Kingwood, TX (US); Christopher D. Smith, Kingwood, TX (US)

(73) Assignee: Basell Poliolefine Italia S.r.l., Milan (IT)

(21) Appl. No.: 19/086,398

(22) Filed: Mar. 21, 2025

Related U.S. Application Data
(60) Provisional application No. 63/568,581, filed on Mar. 22, 2024.
(60) Provisional application No. 63/663,812, filed on Jun. 25, 2024.

(51) Int. Cl. C10G 69/04; C10G 69/06

(52) U.S. Cl. CPC C10G 69/04; C10G 69/06; C10G 2300/1003; C10G 2300/1096; C10G 2300/4081

(57) ABSTRACT

Methods for upgrading heavy aromatic waste streams, such as polystyrene, to chemical feedstocks are disclosed. The process involves feeding waste streams to a first pyrolysis zone followed by hydrogenation. The hydrogenation effluent is processed to recover ethylene, propylene, benzene, ethylbenzene, or their precursors. Optionally, the effluents are separated into light and heavy streams. The light stream from the first pyrolysis effluent is sent to hydrogenation, while the heavy stream is either removed, recycled to the first pyrolysis zone, or fed to a second pyrolysis zone. The light stream from the hydrogenation effluent is processed to recover the same chemicals, and the heavy stream is similarly managed. If present, the second pyrolysis effluent is separated into light and heavy streams, with the light stream processed to recover chemicals and the heavy stream managed as before.

### CROSS-REFERENCE TO RELATED APPLICATIONS

This application claims priority of U.S. Provisional Patent Application Ser. No. 63/568,581, entitled “PROCESS TO UPGRADE AROMATIC WASTE STREAMS,” filed on Mar. 22, 2024, and U.S. Provisional Patent Application Ser. No. 63/663,812, entitled “CATALYTIC CRACKING OF POSM FUEL UPGRADE BOTTOMS (“IRFO”),” filed on Jun. 25, 2024, the contents of which are incorporated by reference herein in their entirety.

### FIELD OF THE INVENTION

This disclosure relates generally to the field of chemical recycling and more specifically to processes for recycling and converting heavy aromatic waste and/or fuel oil streams, including polystyrene and other related materials, back to chemical feedstocks.

### BACKGROUND OF THE INVENTION

The production of styrene and ethylbenzene is a critical aspect of the chemical industry, with applications spanning numerous products, including plastics, resins, and rubber. The Propylene Oxide/Styrene Monomer (POSM) process is a well-known technology for producing styrene and propylene oxide. However, the reliance on non-renewable resources and the generation of by-products and waste streams present environmental and economic challenges.

Polystyrene waste, a significant environmental concern due to its non-biodegradability, has traditionally been addressed through mechanical recycling processes. However, these processes are limited in their ability to achieve complete chemical circularity and often result in downcycling of the material. Furthermore, heavy aromatic waste streams, such as residual fuel oils (RFOs) and heavy aromatic solvents (HAS), are by-products of various chemical processes, including the POSM process itself. These waste streams are often underutilized and pose disposal and environmental issues.

There is a need for improved processes to recycle these waste streams back into valuable chemical feedstocks, thereby reducing reliance on virgin raw materials, enhancing the sustainability of chemical processes, and contributing to the circular economy. Ideally, such improved processes could be implemented in existing facilities by retrofitting or built into new facilities employing commonly used equipment and familiar techniques.

### SUMMARY OF THE INVENTION

In some embodiments, a process to upgrade a heavy aromatic waste stream to chemical feedstocks, comprises subjecting the heavy aromatic waste stream to pyrolysis conditions in one or more pyrolysis reaction zones to produce a first intermediate product having a higher styrene content than the heavy aromatic waste stream and char. The process further comprises subjecting the first intermediate product to hydrogenation conditions in a hydrogenation reaction zone to produce a second intermediate product comprising ethylbenzene. The second intermediate product is then separated to produce a third intermediate product comprising ethylbenzene and a first residual fraction.

In some embodiments, a process to upgrade a heavy aromatic waste stream to chemical feedstocks, comprises subjecting the heavy aromatic waste stream to pyrolysis conditions in one or more pyrolysis reaction zones to produce a first intermediate product and char. The process further comprises separating the first intermediate product to produce a second intermediate product comprising styrene and a first residual fraction. The second intermediate product is then hydrogenated in a second reaction zone to produce a third intermediate product comprising ethylbenzene.

In some embodiments, a process to upgrade a heavy aromatic waste stream to chemical feedstocks, comprises subjecting the heavy aromatic waste stream to pyrolysis conditions in one or more pyrolysis reaction zones to produce a first intermediate product and char. The process further comprises separating the first intermediate product to produce a second intermediate product comprising styrene and a first residual fraction. The second intermediate product is then hydrogenated in a second reaction zone to produce a third intermediate product comprising ethylbenzene. The first residual fraction is subjected to second pyrolysis conditions in one or more second pyrolysis reactions zones to produce a fourth intermediate product. The fourth intermediate product is then separated to produce a fifth intermediate product comprising styrene, benzene, ethylene, propylene, or a combination thereof and a second residual fraction.

The foregoing has outlined rather broadly the features and technical advantages of the present invention in order that the detailed description of the invention that follows may be better understood. Additional features and advantages of the invention will be described hereinafter, which form the subject matter of the claims of the invention. It should be appreciated by those skilled in the art that the conception and specific embodiments disclosed may be readily utilized as a basis for modifying or designing other catalyst compositions and/or processes for carrying out the same purposes of the present invention. It should also be realized by those skilled in the art that such equivalent constructions do not depart from the spirit and scope of the invention as set forth in the appended claims. The novel features which are believed to be characteristic of the invention, both as to its compositions and processes, together with further objects and advantages will be better understood from the following description.

### BRIEF DESCRIPTION OF THE FIGURES

The claimed subject matter may be understood by reference to the following description taken in conjunction with the accompanying drawings, in which like reference numerals identify like elements, and in which:

FIG. 1 is a simplified flow diagram illustrating pyrolysis of heavy aromatic waste feed to produce a first intermediate product having an increased styrene content, followed by hydrogenation of the first intermediate product to convert styrene to ethylbenzene (EB), and separation of the hydrogenated product into a light stream and a heavy stream, and the light stream comprises EB suitable as feedstock to a propylene oxide/styrene monomer (POSM) process or for direct conversion to styrene monomer (SM) via dehydrogenation, according to embodiments of the disclosure;

FIG. 2 is a simplified flow diagram illustrating an extension of the process shown in FIG. 1, wherein the heavy stream is removed from the process, recycled to the first pyrolysis zone, or sent to a second pyrolysis zone to produce additional feedstock to a propylene oxide/styrene monomer (POSM) process or for direct conversion to styrene monomer (SM) via dehydrogenation, according to embodiments of the disclosure;

FIG. 3 is a simplified flow diagram illustrating pyrolysis of heavy aromatic waste feed to produce a first intermediate product having an increased styrene content, separating the first intermediate product into a light stream and a heavy stream, and hydrogenation of the light stream to produce a second intermediate stream comprising EB, suitable as feedstock to a propylene oxide/styrene monomer (POSM) process or for direct conversion to styrene monomer (SM) via dehydrogenation, according to embodiments of the disclosure;

FIG. 4 is a simplified flow diagram illustrating an extension of the process shown in FIG. 3, wherein the heavy stream is removed from the process, recycled to the first pyrolysis zone, or sent to a second pyrolysis zone to produce additional feedstock to a propylene oxide/styrene monomer (POSM) process or for direct conversion to styrene monomer (SM) via dehydrogenation, according to embodiments of the disclosure;

FIG. 5 is a simplified flow diagram illustrating pyrolysis of heavy aromatic waste feed to produce a first intermediate product having an increased styrene content, separating the first intermediate product into a first light stream and a first heavy stream, and hydrogenation of the first light stream to produce a second intermediate stream comprising EB, separation of the second intermediate stream into a second light stream and a second heavy stream, and the second light stream comprises EB suitable as feedstock to propylene oxide/styrene monomer (POSM) process or for direct conversion to styrene monomer (SM) via dehydrogenation, according to embodiments of the disclosure;

FIG. 6 is a simplified flow diagram illustrating an extension of the process shown in FIG. 5, wherein at least a portion of the first and/or second heavy streams are removed from the process, recycled to the first pyrolysis zone, and/or sent to a second pyrolysis zone to produce additional feedstock to a propylene oxide/styrene monomer (POSM) process or for direct conversion to styrene monomer (SM) via dehydrogenation, according to embodiments of the disclosure; and

FIG. 7A and FIG. 7B show a collection of various spectra showing the composition of a fuel oil, according to an embodiment of the disclosure (FIG. 7A), and a series of three MS-GC spectra representing the composition of the catalytically cracked fuel oil (FIG. 7B).

While the disclosed process and composition are susceptible to various modifications and alternative forms, the drawings illustrate specific embodiments herein described in detail by way of example. It should be understood, however, that the description herein of specific embodiments is not intended to limit the invention to the particular forms disclosed, but on the contrary, the intention is to cover all modifications, equivalents, and alternatives falling within the spirit and scope of the invention as defined by the appended claims.

### DETAILED DESCRIPTION OF THE INVENTION

Illustrative embodiments of the subject matter claimed below will now be disclosed. In the interest of clarity, some features of some actual implementations may not be described in this specification. It will be appreciated that in the development of any such actual embodiments, numerous implementation-specific decisions must be made to achieve the developer's specific goals, such as compliance with system-related and business-related constraints, which will vary from one implementation to another. Moreover, it will be appreciated that such a development effort, even if complex and time-consuming, would be a routine undertaking for those of ordinary skill in the art having the benefit of this disclosure.

The words and phrases used herein should be understood and interpreted to have a meaning consistent with the understanding of those words and phrases by those skilled in the relevant art. No special definition of a term or phrase, i.e., a definition that is different from the ordinary and customary meaning as understood by those skilled in the art, is intended to be implied by consistent usage of the term or phrase herein. To the extent that a term or phrase is intended to have a special meaning, i.e., a meaning other than the broadest meaning understood by skilled artisans, such a special or clarifying definition will be expressly set forth in the specification in a definitional manner that provides the special or clarifying definition for the term or phrase.

For example, the following discussion contains a non-exhaustive list of definitions of several specific terms used in this disclosure (other terms may be defined or clarified in a definitional manner elsewhere herein). These definitions are intended to clarify the meanings of the terms used herein. It is believed that the terms are used in a manner consistent with their ordinary meaning, but the definitions are nonetheless specified here for clarity.

### Definitions

As used herein, “a” or “an” when used in conjunction with the term “comprising” in the claims or the specification means one or more than one, unless the context dictates otherwise.

As used herein, “about” means the stated value plus or minus the margin of error of measurement or plus or minus 10% if no process of measurement is indicated.

As used herein, “char” refers to coke, a carbon-containing solid, that accumulates on the catalyst particles during pyrolysis.

As used herein, “consisting essentially of” excludes additional material elements, but allows the inclusions of non-material elements that do not substantially change the nature of the invention.

As used herein, “consisting of” is closed and excludes all additional elements.

As used herein, “conversion” is used to denote the percentage of a component fed which disappears across a reactor.

As used herein, “comprising” is to be interpreted as specifying the presence of the stated features, integers, steps, or components as referred to, but does not preclude the presence or addition of one or more features, integers, steps, or components, or groups thereof. Moreover, each of the terms “by”, “comprising,” “comprises”, “comprised of” “including,” “includes,” “included,” “involving,” “involves,” “involved,” and “such as” are used in their open, non-limiting sense and may be used interchangeably. Further, the term “comprising” is intended to include examples and aspects encompassed by the terms “consisting essentially of” and “consisting of.” Similarly, the term “consisting essentially of” is intended to include examples encompassed by the term “consisting of.”

As used herein, “reaction zone” refers to a chamber sufficiently enclosed to maintain selected operating conditions within the chamber to produce a desired reaction, such as a pyrolysis reaction zone, a steam cracking reaction zone, a catalytic cracking reaction zone, or a hydrogenation reaction zone. In some embodiments, each reaction zone can be a separate reactor. In some embodiments, a single vessel can contain a plurality of reaction zones.

As used herein, “separation conditions” or “separation section” means facilities including distillation, absorption, adsorption, membrane separation, cryogenic distillation, extractive distillation, azeotropic distillation, steam distillation, molecular sieves, liquid-liquid extraction (solvent extraction), decantation, centrifugation, or a combination thereof as required to recover specific materials from intermediate products and/or reaction products disclosed herein. Such embodiments would include common equipment associated with the forgoing separation processes, including, but not limited to, columns, drums, vessels, heat exchangers, pumps, valves, reflux loops, and the like, the descriptions of which are omitted herein for simplicity. Where intermediate products and/or reaction products disclosed herein are defined as comprising multiple products (e.g., benzene, ethylene, propylene, or a combination thereof), it is intended that “separation conditions” or “separation section” includes the functionality to recover each of those products separately at a desired purity (e.g., 99 wt % benzene, 99.5 wt % ethylene, 97 wt % propylene, etc.) as required by the disposition of the product as a feed to various locations of other processes, such as, but not limited to, POSM and/or SM.

As used herein, “waste stream” is a type of feed stream comprising material that has been discarded as no longer useful, including but not limited to, post-consumer and post-industrial waste.

As used herein, “zeolite” refers to an aluminosilicate mineral with a microporous structure. Zeolites are, in one aspect, useful as catalysts for the processes disclosed herein. Zeolites can occur naturally or can be produced industrially.

All concentrations herein are by weight percent (“wt %”) unless otherwise specified.

The following abbreviations are used herein:

| Abbreviation | Term |
|---|---|
| ECR | ethylene cracking residue |
| HAS | heavy aromatic solvent |
| PAH | polycyclic aromatic hydrocarbons |
| PFO | pyrolysis fuel oil |
| POSM | Propylene oxide/styrene monomer production process |
| RFO | residual fuel oils |
| SM | Styrene monomer or process to produce styrene monomer, depending |
| | on the context |
| WHSV | weight hourly space velocity |

### Heavy Aromatic Waste

The present disclosure provides a process for upgrading aromatic waste, including polystyrene, into light olefins (e.g., ethylene and/or propylene), ethylbenzene, and/or ethylbenzene precursors useful as feedstocks to petrochemical processes, including, but not limited to, the propylene oxide/styrene monomer process and dehydrogenation, oxidative or non-oxidative, of ethylbenzene to produce styrene. Heavy aromatic waste streams include, but are not limited to, polystyrene pyrolysis oil, styrene, polystyrene pyrolysis oil heavies, heavy residual fuel oils (RFO), heavy aromatic solvent (HAS), pyrolysis fuel oil (PFO), and ethylene cracking residue (ECR). Heavy aromatic waste streams to be treated by the processes disclosed herein can be any one of the foregoing materials or any mixture of two or more of the foregoing materials.

Polystyrene pyrolysis involves the thermal degradation of polystyrene, a common thermoplastic material, in the absence of oxygen. This process breaks down the long polymer chains of polystyrene back into shorter hydrocarbon chains and styrene monomer, along with a range of other hydrocarbons. The products of polystyrene pyrolysis can be broadly categorized into two groups: polystyrene pyrolysis oil and polystyrene pyrolysis oil heavies. Each of these categories has distinct characteristics and potential applications. Polystyrene pyrolysis oil primarily consists of styrene monomer, which can be recovered and purified for reuse in the production of new polystyrene products or other styrenic polymers. Besides styrene, the oil contains a mixture of other aromatic hydrocarbons such as toluene, ethylbenzene, and benzene, as well as aliphatic hydrocarbons. The exact composition of the pyrolysis oil can vary based on the pyrolysis conditions such as temperature, heating rate, and the presence of catalysts. Polystyrene pyrolysis oil heavies consist of the heavier, more complex hydrocarbons that are produced during the pyrolysis process. These compounds are higher in molecular weight compared to the main fraction of pyrolysis oil and often include polycyclic aromatic hydrocarbons (PAH), along with various oligomers formed by partial recombination of degradation products. Pyrolysis of polystyrene presents an opportunity for recycling a plastic that is otherwise difficult to process through mechanical recycling methods. By converting waste polystyrene into valuable chemicals and fuels, pyrolysis can reduce landfilling and incineration, contributing to circular economy initiatives.

During the POSM process, various by-products are generated, including residual fuel oils (RFO) rich in aromatics. These RFOs are complex mixtures containing high molecular weight hydrocarbons, predominantly aromatics, along with aliphatics and small amounts of olefins. The aromatic content gives these oils their distinct characteristics, including high density and a high calorific value. The exact composition of these oils can vary depending on the specifics of the POSM process and the feedstocks used. These oils have high boiling points due to the presence of large, complex hydrocarbon molecules. The high aromatic content contributes to a higher density and viscosity compared to lighter fuel oils. While these oils are rich in energy content, their high aromatic content may affect their combustion characteristics. Depending on the feedstock and process conditions, the sulfur content can vary, potentially requiring desulfurization treatments for certain uses. Such RFOs have utility as components in fuel oil and asphalt blending but incur some safety, environmental, and regulatory considerations due to high aromatics content and/or emissions such as NOx, SOx, and particulate matter.

Benzene is typically converted to ethylbenzene by reacting benzene with ethylene in an alkylation process. Heavy aromatic solvents (HAS) can be produced as by-products of the alkylation process. These heavy aromatic solvents are generally composed of higher molecular weight aromatic compounds that form through side reactions during the alkylation process. A common HAS produced in this context is diethylbenzene (DEB), which consists of three isomers: ortho-, meta-, and para-diethylbenzene. Diethylbenzene forms when an ethyl group is added to ethylbenzene in a subsequent alkylation reaction, essentially representing an over-alkylation of benzene. Although DEB has some direct uses, it would be desirable to break down and convert these heavy molecules into basic monomers suitable as feedstocks to a variety of petrochemical processes, including, but not limited to, POSM and EB dehydrogenation to SM.

In steam cracking processes, complex mixtures of by-products are generated in addition to producing olefins (e.g., ethylene, propylene). Such by-products include heavy aromatic-rich higher molecular weight hydrocarbons such as pyrolysis fuel oil (PFO) and ethylene cracking residue (ECR). PFO is a complex mixture that includes heavy aromatics, asphaltenes, and other hydrocarbons produced during the thermal cracking of naphtha or gas oils. Its exact composition varies depending on the feedstock and the operating conditions of the cracker but is characterized by its high content of polycyclic aromatic hydrocarbons (PAHs). ECR is the residue left from the steam cracking process, particularly when heavier feedstocks are used. It is rich in heavy aromatics, tar, and coke precursors. Like PFO, its specific composition depends on the feedstock and process conditions. Similarly to RFOs, PFO and ECR have utility as components in fuel oil but incur some safety, environmental, and regulatory considerations due to high aromatics content and/or emissions such as NOx, SOx, and particulate matter.

The present disclosure provides embodiments of processes to upgrade heavy aromatic waste streams, including polystyrene and other related materials, to chemical feedstocks. In some embodiments, the heavy aromatic waste stream comprises polystyrene pyrolysis oil, styrene, polystyrene pyrolysis oil heavies, heavy residual fuel oils (RFO), heavy aromatic solvent (HAS), pyrolysis fuel oil (PFO), ethylene cracking residue (ECR), or a combination thereof. In some embodiments, the heavy aromatic waste stream comprises styrene in an amount greater than or equal to 30 wt %, greater than or equal to 40 wt %, greater than or equal to 50 wt %, or greater than or equal to 60 wt %, based on the total weight of the heavy aromatic waste stream.

In some embodiments, the present disclosure provides a method for converting a fuel oil to propylene comprising: (a) providing a fuel oil comprising a plurality of aromatic compounds comprising: (i) from about 1.0 wt % to about 2.0 wt % ethylbenzene; and (ii) from about 1.0 wt % to about 2.0 wt % styrene, wherein the fuel oil comprises no more than 1.0 wt % benzene and 0.2 wt % propylene; and (b) catalytically cracking the fuel oil in the presence of a catalyst to produce a cracked product comprising: (iii) from about 10 wt % to about 19 wt % propylene; and (iv) from about 36 wt % to about 50 wt % benzene.

### Process Comprising Pyrolysis/Hydrogenation

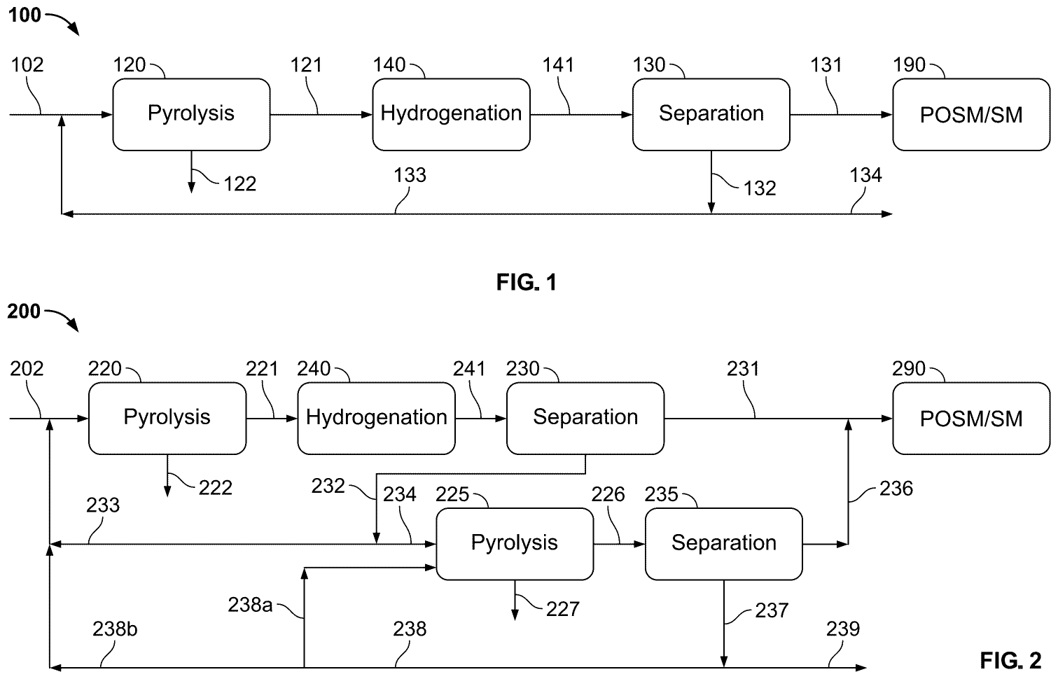

FIG. 1 shows a process 100 to upgrade a heavy aromatic waste stream 502 into chemical feedstocks. The heavy aromatic waste stream 102 has a first styrene content. The process conditions and catalyst selection are optimized to achieve a desired product composition. In some embodiments, the process 100 comprises subjecting the heavy aromatic waste stream 102 to pyrolysis conditions in one or more pyrolysis reaction zones 120 to produce a first intermediate product 121 having a higher styrene content than the heavy aromatic waste stream and char 122.

The pyrolysis conditions and the one or more pyrolysis reaction zones 120 comprise thermal pyrolysis conditions in one or more thermal pyrolysis reaction zones, catalytic pyrolysis conditions in one or more catalytic pyrolysis zones, a combination thereof.

The thermal pyrolysis conditions comprise:
a) a temperature in the range of from about 200° C. to about 800° C., from 300° C. to 700° C., or from 400° C. to 600° C.;
b) a pressure in the range of from 5 kPa (absolute) to 450 kPa, from 13.3 kPa (absolute) to 400 kPa, from atmospheric pressure to 350 kPa, or from 100 kPa to 300 kPa; or
c) a combination thereof.

The catalytic pyrolysis conditions comprise:
a) a temperature in the range of from 300° C. to 600° C., from 250° C. to 450° C., from 280° C. to 420° C., or from 280° C. to 350° C.;
b) a pressure in the range of from 5 kPa (absolute) to 450 kPa, from 13.3 kPa (absolute) to 400 kPa, from atmospheric pressure to 350 kPa, or from 100 kPa to 300 kPa;
c) a weight hourly space velocity in the range of from 0.1 hr⁻¹ to 100 hr⁻¹, from 0.5 hr⁻¹ to 60 hr⁻¹, from 0.8 hr⁻¹ to 25 hr⁻¹, from 1.0 hr⁻¹ to 25 hr⁻¹, or from 3.0 hr⁻¹ to 25 hr⁻¹; or
d) a combination thereof.

In some embodiments, catalytic cracking the fuel oil occurs at a temperature greater than about 380° C. In some embodiments, catalytic cracking the fuel oil occurs at a temperature greater than about 480° C. In some embodiments, catalytic cracking the fuel oil occurs at a temperature greater than about 580° C. In some embodiments, catalytic cracking the fuel oil occurs at a temperature in the range of from about 350° C. to about 650° C. In some embodiments, catalytic cracking the fuel oil occurs at a temperature in the range of from about 350° C. to about 450° C. In some embodiments, catalytic cracking the fuel oil occurs at a temperature in the range of from about 450° C. to about 550° C. In some embodiments, catalytic cracking the fuel oil occurs at a temperature in the range of from about 550° C. to about 650° C. In some embodiments, the catalyst is a zeolite. In some embodiments, the catalyst is a H-ZSM-5 zeolite. In some embodiments, the fuel oil is obtained from a propylene oxide/styrene monomer (POSM) production process.

The catalytic pyrolysis conditions further comprise a medium or large pore zeolite or molecular sieve. The medium or large pore zeolite or molecular sieve comprises beta zeolite (BEA structure), ferrierite (FER structure), mordenite (MOR structure), zeolite L (LTL structure), zeolite X (FAU structure), zeolite Y (FAU structure), ZSM-11 (MEL structure), ZSM-22 (TON structure), ZSM-23 (MTT structure), ZSM-5 (MFI structure), or a combination thereof.

In some embodiments, pyrolysis section 120 is configured to provide one or more pyrolysis reaction zones in series. In some embodiments wherein the one or more pyrolysis reaction zones in series, a reaction product of at least one pyrolysis reaction zone is separated into an intermediate product stream and a residual fraction, and at least one of the intermediate product stream and the residual fraction is fed to a downstream pyrolysis reaction zone. In some embodiments, pyrolysis section 120 comprises one or more thermal pyrolysis zones. In some embodiments, pyrolysis section 120 comprises one or more catalytic pyrolysis zones. In some embodiments, pyrolysis section 120 comprises at least one thermal pyrolysis zone followed by at least one catalytic pyrolysis zone. In some embodiments, pyrolysis section 120 comprises at least one catalytic pyrolysis zone followed by at least one thermal pyrolysis zone. In some embodiments, pyrolysis section 120 comprises two or more pyrolysis zones in series (e.g., thermal pyrolysis/thermal pyrolysis, thermal pyrolysis/catalytic pyrolysis, catalytic pyrolysis/thermal pyrolysis, catalytic pyrolysis/catalytic pyrolysis). In some embodiments, pyrolysis section 120 comprises an upstream pyrolysis zone followed by a separation system followed by a downstream pyrolysis zone. The separation system separates the upstream pyrolysis effluent into a light stream and a heavy stream. The light stream is sent as feed to the downstream pyrolysis zone and the heavy stream is recycled to the upstream pyrolysis zone as additional feed.

The first intermediate product 121 is subjected to hydrogenation conditions in hydrogenation section 140 to produce second intermediate product 141. The second intermediate product 141 is subjected to separation conditions in separation section 130 to produce third intermediate product 131 and first residual fraction 132. Examples of hydrogenation catalysts include any commercial hydrogenation catalysts known in the art such as NiMo, CoMo, Ni, Pd, Pt, Cu, and the like.

In some embodiments, the ratio of the styrene content of the first intermediate product 121 to the styrene content of the heavy aromatic waste stream 102 is greater than or equal to 1.01, greater than or equal to 1.02, greater than or equal to 1.05, greater than or equal to 1.10, greater than or equal to 1.15, or greater than or equal to 1.20.

In some embodiments, the second intermediate product 141 comprises ethylbenzene in an amount greater than or equal to 30 wt %, greater than or equal to 40 wt %, greater than or equal to 50 wt %, or greater than or equal to 60 wt %, based on the total weight of the second intermediate product.

In some embodiments, the third intermediate product 131 comprises ethylbenzene in an amount greater than or equal to 60 wt %, greater than or equal to 70 wt %, greater than or equal to 80 wt %, greater than or equal to 90 wt %, greater than or equal to 95 wt %, greater than or equal to 98 wt %, greater than or equal to 99 wt %, greater than or equal to 99.5 wt %, or greater than or equal to 99.9 wt %, based on the total weight of the third intermediate product.

The third intermediate product 131 is then fed to a POSM/SM process 190, wherein POSM/SM means the POSM process and/or the SM process. SM process means the direct dehydrogenation of EB to form styrene monomer. The third intermediate product 131 comprises EB. As part of POSM/SM process 190, EB is recovered from the third intermediate product 131, wherein the purity of the recovered EB is suitable for direct or indirect addition to the POSM and/or the SM process. Indirect addition means that one or more additional processing steps can be performed on the recovered stream to make it suitable for addition to the POSM and/or the SM process. The specific components in the heavy aromatic waste stream 102, the specific pyrolysis conditions in the pyrolysis section 120, the specific hydrogenation conditions in the hydrogenation section 140, and the specific separation conditions in the separation section 130 will result in different amounts of styrene in the third intermediate product 131. Examples of hydrogenation catalysts include any commercial hydrogenation catalysts known in the art such as NiMo, CoMo, Ni, Pd, Pt, Cu, and the like.

In some embodiments, ethylbenzene is recovered from the third intermediate product 131. In some embodiments, at least a portion of the EB is added to the POSM process, wherein EB is oxidized to form ethylbenzene hydroperoxide. The ethylbenzene hydroperoxide is then catalytically reacted the with propylene to form propylene oxide and 1-phenyl ethanol. The 1-phenyl ethanol is then dehydrated to produce styrene monomer. In some embodiments, at least a portion of the EB is added to the SM process, wherein EB is dehydrogenated to produce styrene monomer. In some embodiments, EB is added to both the POSM and SM processes.

In some embodiments, the first residual fraction 132 is added to the pyrolysis section 120 as additional feed via stream 133, sent outside the process for addition processing via stream 134, or a combination thereof.

FIG. 2 shows a process 200 to upgrade a heavy aromatic waste stream 502 into chemical feedstocks. The heavy aromatic waste stream 202 has a first styrene content. The process conditions and catalyst selection are optimized to achieve a desired product composition. In some embodiments, the process 200 comprises subjecting the heavy aromatic waste stream 202 to pyrolysis conditions in one or more pyrolysis reaction zones 220 to produce a first intermediate product 221 having a higher styrene content than the heavy aromatic waste stream and char 222.

The pyrolysis conditions and the one or more pyrolysis reaction zones 220 comprise thermal pyrolysis conditions in one or more thermal pyrolysis reaction zones, catalytic pyrolysis conditions in one or more catalytic pyrolysis zones, a combination thereof.

The thermal pyrolysis conditions comprise:
a) a temperature in the range of from about 200° C. to about 800° C., from 300° C. to 700° C., or from 400° C. to 600° C.;
b) a pressure in the range of from 5 kPa (absolute) to 450 kPa, from 13.3 kPa (absolute) to 400 kPa, from atmospheric pressure to 350 kPa, or from 100 kPa to 300 kPa; or
c) a combination thereof.

The catalytic pyrolysis conditions comprise:
a) a temperature in the range of from 300° C. to 600° C., from 250° C. to 450° C., from 280° C. to 420° C., or from 280° C. to 350° C.;
b) a pressure in the range of from 5 kPa (absolute) to 450 kPa, from 13.3 kPa (absolute) to 400 kPa, from atmospheric pressure to 350 kPa, or from 100 kPa to 300 kPa;
c) a weight hourly space velocity in the range of from 0.1 hr⁻¹ to 100 hr⁻¹, from 0.5 hr⁻¹ to 60 hr⁻¹, from 0.8 hr⁻¹ to 25 hr⁻¹, from 1.0 hr⁻¹ to 25 hr⁻¹, or from 3.0 hr⁻¹ to 25 hr⁻¹; or
d) a combination thereof.

In some embodiments, catalytic cracking the fuel oil occurs at a temperature greater than about 380° C. In some embodiments, catalytic cracking the fuel oil occurs at a temperature greater than about 480° C. In some embodiments, catalytic cracking the fuel oil occurs at a temperature greater than about 580° C. In some embodiments, catalytic cracking the fuel oil occurs at a temperature in the range of from about 350° C. to about 650° C. In some embodiments, catalytic cracking the fuel oil occurs at a temperature in the range of from about 350° C. to about 450° C. In some embodiments, catalytic cracking the fuel oil occurs at a temperature in the range of from about 450° C. to about 550° C. In some embodiments, catalytic cracking the fuel oil occurs at a temperature in the range of from about 550° C. to about 650° C. In some embodiments, the catalyst is a zeolite. In some embodiments, the catalyst is a H-ZSM-5 zeolite. In some embodiments, the fuel oil is obtained from a propylene oxide/styrene monomer (POSM) production process.

The catalytic pyrolysis conditions further comprise a medium or large pore zeolite or molecular sieve. The medium or large pore zeolite or molecular sieve comprises beta zeolite (BEA structure), ferrierite (FER structure), mordenite (MOR structure), zeolite L (LTL structure), zeolite X (FAU structure), zeolite Y (FAU structure), ZSM-11 (MEL structure), ZSM-22 (TON structure), ZSM-23 (MTT structure), ZSM-5 (MFI structure), or a combination thereof.

In some embodiments, pyrolysis section 220 is configured to provide one or more pyrolysis reaction zones in series. In some embodiments wherein the one or more pyrolysis reaction zones in series, a reaction product of at least one pyrolysis reaction zone is separated into an intermediate product stream and a residual fraction, and at least one of the intermediate product stream and the residual fraction is fed to a downstream pyrolysis reaction zone.

In some embodiments, pyrolysis section 220 comprises one or more thermal pyrolysis zones. In some embodiments, pyrolysis section 220 comprises one or more catalytic pyrolysis zones. In some embodiments, pyrolysis section 220 comprises at least one thermal pyrolysis zone followed by at least one catalytic pyrolysis zone. In some embodiments, pyrolysis section 220 comprises at least one catalytic pyrolysis zone followed by at least one thermal pyrolysis zone. In some embodiments, pyrolysis section 220 comprises two or more pyrolysis zones in series (e.g., thermal pyrolysis/thermal pyrolysis, thermal pyrolysis/catalytic pyrolysis, catalytic pyrolysis/thermal pyrolysis, catalytic pyrolysis/catalytic pyrolysis). In some embodiments, pyrolysis section 220 comprises an upstream pyrolysis zone followed by a separation system followed by a downstream pyrolysis zone. The separation system separates the upstream pyrolysis effluent into a light stream and a heavy stream. The light stream is sent as feed to the downstream pyrolysis zone and the heavy stream is recycled to the upstream pyrolysis zone as additional feed.

The first intermediate product 221 is subjected to hydrogenation conditions in hydrogenation section 240 to produce second intermediate product 241. The second intermediate product 241 is subjected to separation conditions in separation section 230 to produce third intermediate product 231 and first residual fraction 232. Examples of hydrogenation catalysts include any commercial hydrogenation catalysts known in the art such as NiMo, CoMo, Ni, Pd, Pt, Cu, and the like.

In some embodiments, the ratio of the styrene content of the first intermediate product 221 to the styrene content of the heavy aromatic waste stream 202 is greater than or equal to 1.01, greater than or equal to 1.02, greater than or equal to 1.05, greater than or equal to 1.10, greater than or equal to 1.15, or greater than or equal to 1.20.

In some embodiments, the second intermediate product 241 comprises ethylbenzene in an amount greater than or equal to 30 wt %, greater than or equal to 40 wt %, greater than or equal to 50 wt %, or greater than or equal to 60 wt %, based on the total weight of the second intermediate product.

In some embodiments, the third intermediate product 231 comprises ethylbenzene in an amount greater than or equal to 60 wt %, greater than or equal to 70 wt %, greater than or equal to 80 wt %, greater than or equal to 90 wt %, greater than or equal to 95 wt %, greater than or equal to 98 wt %, greater than or equal to 99 wt %, greater than or equal to 99.5 wt %, or greater than or equal to 99.9 wt %, based on the total weight of the third intermediate product.

The third intermediate product 231 is then fed to a POSM/SM process 290, wherein POSM/SM means the POSM process and/or the SM process. SM process means the direct dehydrogenation of EB to form styrene monomer. The third intermediate product 231 comprises EB. As part of POSM/SM process 290, EB is recovered from the third intermediate product 231, wherein the purity of the recovered EB is suitable for direct or indirect addition to the POSM and/or the SM process. Indirect addition means that one or more additional processing steps can be performed on the recovered stream to make it suitable for addition to the POSM and/or the SM process. The specific components in the heavy aromatic waste stream 202, the specific pyrolysis conditions in the pyrolysis section 220, the specific hydrogenation conditions in the hydrogenation section 240, and the specific separation conditions in the separation section 230 will result in different amounts of styrene in the third intermediate product 231. Examples of hydrogenation catalysts include any commercial hydrogenation catalysts known in the art such as NiMo, CoMo, Ni, Pd, Pt, Cu, and the like.

In some embodiments, ethylbenzene is recovered from the third intermediate product 231. In some embodiments, at least a portion of the EB is added to the POSM process, wherein EB is oxidized to form ethylbenzene hydroperoxide. The ethylbenzene hydroperoxide is then catalytically reacted the with propylene to form propylene oxide and 1-phenyl ethanol. The 1-phenyl ethanol is then dehydrated to produce styrene monomer. In some embodiments, at least a portion of the EB is added to the SM process, wherein EB is dehydrogenated to produce styrene monomer. In some embodiments, EB is added to both the POSM and SM processes.

In some embodiments, ethylbenzene is recovered from the third intermediate product 241. In some embodiments, at least a portion of the EB is added to the POSM process, wherein EB is oxidized to form ethylbenzene hydroperoxide. The ethylbenzene hydroperoxide is then catalytically reacted the with propylene to form propylene oxide and 1-phenyl ethanol. The 1-phenyl ethanol is then dehydrated to produce styrene monomer. In some embodiments, at least a portion of the EB is added to the SM process, wherein EB is dehydrogenated to produce styrene monomer. In some embodiments, EB is added to both the POSM and SM processes.

In some embodiments, the first residual fraction 232 is added to a second the pyrolysis section 225 as feed via stream 434, added to a first the pyrolysis section 220 as additional feed via stream 233, or a combination thereof. First residual fraction 234 is subjected to pyrolysis conditions in one or more pyrolysis reaction zones 225 to produce a fourth intermediate product 226 having a higher styrene content than the first residual fraction 232 and char 227.

The pyrolysis conditions and the one or more pyrolysis reaction zones 225 comprise thermal pyrolysis conditions in one or more thermal pyrolysis reaction zones, catalytic pyrolysis conditions in one or more catalytic pyrolysis zones, a combination thereof.

The thermal pyrolysis conditions comprise:
a) a temperature in the range of from about 200° C. to about 800° C., from 300° C. to 700° C., or from 400° C. to 600° C.;
b) a pressure in the range of from 5 kPa (absolute) to 450 kPa, from 13.3 kPa (absolute) to 400 kPa, from atmospheric pressure to 350 kPa, or from 100 kPa to 300 kPa; or
c) a combination thereof.

The catalytic pyrolysis conditions comprise:
a) a temperature in the range of from 300° C. to 600° C., from 250° C. to 450° C., from 280° C. to 420° C., or from 280° C. to 350° C.;
b) a pressure in the range of from 5 kPa (absolute) to 450 kPa, from 13.3 kPa (absolute) to 400 kPa, from atmospheric pressure to 350 kPa, or from 100 kPa to 300 kPa;
c) a weight hourly space velocity in the range of from 0.1 hr⁻¹ to 100 hr⁻¹, from 0.5 hr⁻¹ to 60 hr⁻¹, from 0.8 hr⁻¹ to 25 hr⁻¹, from 1.0 hr⁻¹ to 25 hr⁻¹, or from 3.0 hr⁻¹ to 25 hr⁻¹; or
d) a combination thereof.

In some embodiments, catalytic cracking the fuel oil occurs at a temperature greater than about 380° C. In some embodiments, catalytic cracking the fuel oil occurs at a temperature greater than about 480° C. In some embodiments, catalytic cracking the fuel oil occurs at a temperature greater than about 580° C. In some embodiments, catalytic cracking the fuel oil occurs at a temperature in the range of from about 350° C. to about 650° C. In some embodiments, catalytic cracking the fuel oil occurs at a temperature in the range of from about 350° C. to about 450° C. In some embodiments, catalytic cracking the fuel oil occurs at a temperature in the range of from about 450° C. to about 550° C. In some embodiments, catalytic cracking the fuel oil occurs at a temperature in the range of from about 550° C. to about 650° C. In some embodiments, the catalyst is a zeolite. In some embodiments, the catalyst is a H-ZSM-5 zeolite. In some embodiments, the fuel oil is obtained from a propylene oxide/styrene monomer (POSM) production process.

The catalytic pyrolysis conditions further comprise a medium or large pore zeolite or molecular sieve. The medium or large pore zeolite or molecular sieve comprises beta zeolite (BEA structure), ferrierite (FER structure), mordenite (MOR structure), zeolite L (LTL structure), zeolite X (FAU structure), zeolite Y (FAU structure), ZSM-11 (MEL structure), ZSM-22 (TON structure), ZSM-23 (MTT structure), ZSM-5 (MFI structure), or a combination thereof.

In some embodiments, pyrolysis section 225 is configured to provide one or more pyrolysis reaction zones in series. In some embodiments wherein the one or more pyrolysis reaction zones in series, a reaction product of at least one pyrolysis reaction zone is separated into an intermediate product stream and a residual fraction, and at least one of the intermediate product stream and the residual fraction is fed to a downstream pyrolysis reaction zone.

In some embodiments, pyrolysis section 225 comprises one or more thermal pyrolysis zones. In some embodiments, pyrolysis section 225 comprises one or more catalytic pyrolysis zones. In some embodiments, pyrolysis section 225 comprises at least one thermal pyrolysis zone followed by at least one catalytic pyrolysis zone. In some embodiments, pyrolysis section 225 comprises at least one catalytic pyrolysis zone followed by at least one thermal pyrolysis zone. In some embodiments, pyrolysis section 225 comprises two or more pyrolysis zones in series (e.g., thermal pyrolysis/thermal pyrolysis, thermal pyrolysis/catalytic pyrolysis, catalytic pyrolysis/thermal pyrolysis, catalytic pyrolysis/catalytic pyrolysis). In some embodiments, pyrolysis section 225 comprises an upstream pyrolysis zone followed by a separation system followed by a downstream pyrolysis zone. The separation system separates the upstream pyrolysis effluent into a light stream and a heavy stream. The light stream is sent as feed to the downstream pyrolysis zone and the heavy stream is recycled to the upstream pyrolysis zone as additional feed.

The fourth intermediate product 226 is subjected to separation conditions in separation section 235 to produce fifth intermediate product 236 and second residual fraction 237.

In some embodiments, the ratio of the styrene content of the fourth intermediate product 226 to the styrene content of the first residual fraction 234 is greater than or equal to 1.01, greater than or equal to 1.02, greater than or equal to 1.05, greater than or equal to 1.10, greater than or equal to 1.15, or greater than or equal to 1.20.

In some embodiments, the fifth intermediate product 236 comprises ethylbenzene in an amount greater than or equal to 30 wt %, greater than or equal to 40 wt %, greater than or equal to 50 wt %, or greater than or equal to 60 wt %, based on the total weight of the second intermediate product.

The fifth intermediate product 236 is then fed to a POSM/SM process 290, wherein POSM/SM means the POSM process and/or the SM process. SM process means the direct dehydrogenation of EB to form styrene monomer. The fifth intermediate product 236 comprises one or more of styrene, benzene, ethylene, and propylene. As part of POSM/SM process 290, one or more of styrene, benzene, ethylene, and propylene is recovered from the fifth intermediate product 236, wherein the purity of the recovered styrene, benzene, ethylene, and propylene is suitable for direct or indirect addition to the POSM and/or the SM process. Indirect addition means that one or more additional processing steps can be performed on the recovered stream to make it suitable for addition to the POSM and/or the SM process. The specific components in the first residual fraction 234, the specific pyrolysis conditions in the pyrolysis reaction zone 225, and the specific separation conditions in the separation section 235 will result in different amounts of styrene, benzene, ethylene, and/or propylene in the fifth intermediate product 236.

In some embodiments, styrene is recovered from the fifth intermediate product 236. The styrene is hydrogenated to form EB. Examples of hydrogenation catalysts include any commercial hydrogenation catalysts known in the art such as NiMo, CoMo, Ni, Pd, Pt, Cu, and the like. In some embodiments, at least a portion of the EB is added to the POSM process, wherein EB is oxidized to form ethylbenzene hydroperoxide. The ethylbenzene hydroperoxide is then catalytically reacted the with propylene to form propylene oxide and 1-phenyl ethanol. The 1-phenyl ethanol is then dehydrated to produce styrene monomer. In some embodiments, at least a portion of the EB is added to the SM process, wherein EB is dehydrogenated to produce styrene monomer. In some embodiments, EB is added to both the POSM and SM processes.

In some embodiments, benzene is recovered from the fifth intermediate product 236. The benzene is reacted with ethylene to form EB. In some embodiments, at least a portion of the EB is added to the POSM process, wherein EB is oxidized to form ethylbenzene hydroperoxide. The ethylbenzene hydroperoxide is then catalytically reacted the with propylene to form propylene oxide and 1-phenyl ethanol. The 1-phenyl ethanol is then dehydrated to produce styrene monomer. In some embodiments, at least a portion of the EB is added to the SM process, wherein EB is dehydrogenated to produce styrene monomer. In some embodiments, EB is added to both the POSM and SM processes.

In some embodiments, ethylene is recovered from the fifth intermediate product 236. The ethylene is reacted with benzene to form EB. In some embodiments, at least a portion of the EB is added to the POSM process, wherein EB is oxidized to form ethylbenzene hydroperoxide. The ethylbenzene hydroperoxide is then catalytically reacted the with propylene to form propylene oxide and 1-phenyl ethanol. The 1-phenyl ethanol is then dehydrated to produce styrene monomer. In some embodiments, at least a portion of the EB is added to the SM process, wherein EB is dehydrogenated to produce styrene monomer. In some embodiments, EB is added to both the POSM and SM processes.

In some embodiments, propylene is recovered from the fifth intermediate product 236. The propylene is added to the POSM process, wherein it is reacted with ethylbenzene hydroperoxide to form propylene oxide and 1-phenyl ethanol. The 1-phenyl ethanol is then dehydrated to produce styrene monomer.

In some embodiments, the second residual fraction 237 is added to the pyrolysis section 220 as additional feed via streams 238, 238b, added to the pyrolysis section 225 as additional feed via streams 238, 238a, sent outside the process for addition processing via stream 239, or a combination thereof.

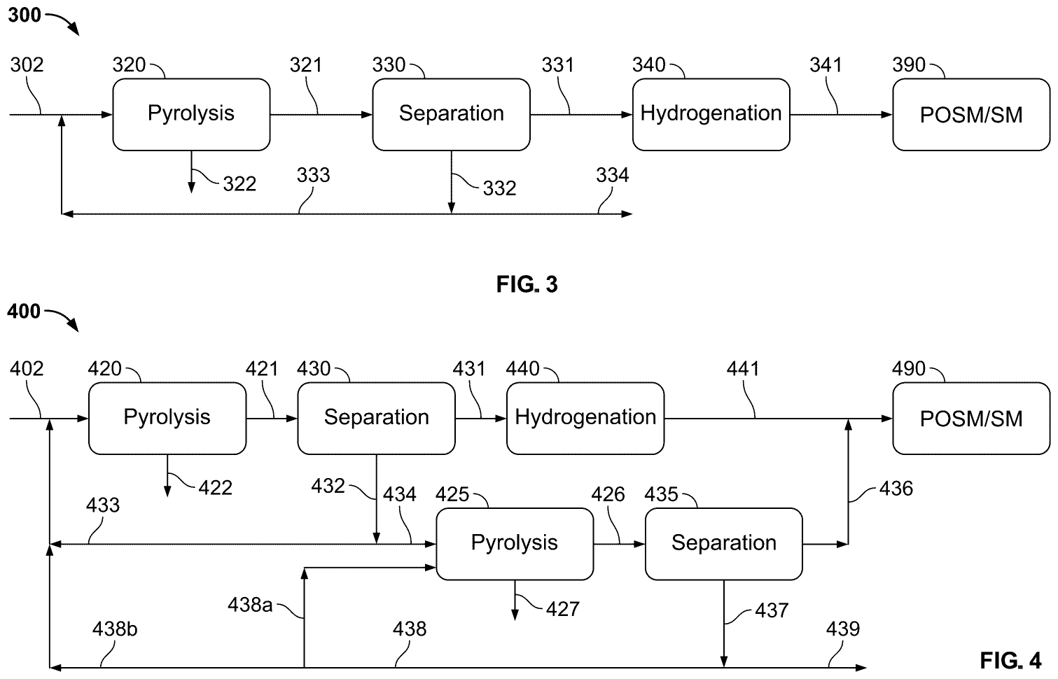

FIG. 3 shows a process 300 to upgrade a heavy aromatic waste stream 302 into chemical feedstocks. The heavy aromatic waste stream 302 has a first styrene content. The process conditions and catalyst selection are optimized to achieve a desired product composition. In some embodiments, the process 300 comprises subjecting the heavy aromatic waste stream 302 to pyrolysis conditions in one or more pyrolysis reaction zones 320 to produce a first intermediate product 321 having a higher styrene content than the heavy aromatic waste stream and char 322.

The pyrolysis conditions and the one or more pyrolysis reaction zones 320 comprise thermal pyrolysis conditions in one or more thermal pyrolysis reaction zones, catalytic pyrolysis conditions in one or more catalytic pyrolysis zones, a combination thereof.

The thermal pyrolysis conditions comprise:
a) a temperature in the range of from about 200° C. to about 800° C., from 300° C. to 700° C., or from 400° C. to 600° C.;
b) a pressure in the range of from 5 kPa (absolute) to 450 kPa, from 13.3 kPa (absolute) to 400 kPa, from atmospheric pressure to 350 kPa, or from 100 kPa to 300 kPa; or
c) a combination thereof.

The catalytic pyrolysis conditions comprise:
a) a temperature in the range of from 300° C. to 600° C., from 250° C. to 450° C., from 280° C. to 420° C., or from 280° C. to 350° C.;
b) a pressure in the range of from 5 kPa (absolute) to 450 kPa, from 13.3 kPa (absolute) to 400 kPa, from atmospheric pressure to 350 kPa, or from 100 kPa to 300 kPa;
c) a weight hourly space velocity in the range of from 0.1 hr⁻¹ to 100 hr⁻¹, from 0.5 hr⁻¹ to 60 hr⁻¹, from 0.8 hr⁻¹ to 25 hr⁻¹, from 1.0 hr⁻¹ to 25 hr⁻¹, or from 3.0 hr⁻¹ to 25 hr⁻¹; or
d) a combination thereof.

In some embodiments, catalytic cracking the fuel oil occurs at a temperature greater than about 380° C. In some embodiments, catalytic cracking the fuel oil occurs at a temperature greater than about 480° C. In some embodiments, catalytic cracking the fuel oil occurs at a temperature greater than about 580° C. In some embodiments, catalytic cracking the fuel oil occurs at a temperature in the range of from about 350° C. to about 650° C. In some embodiments, catalytic cracking the fuel oil occurs at a temperature in the range of from about 350° C. to about 450° C. In some embodiments, catalytic cracking the fuel oil occurs at a temperature in the range of from about 450° C. to about 550° C. In some embodiments, catalytic cracking the fuel oil occurs at a temperature in the range of from about 550° C. to about 650° C. In some embodiments, the catalyst is a zeolite. In some embodiments, the catalyst is a H-ZSM-5 zeolite. In some embodiments, the fuel oil is obtained from a propylene oxide/styrene monomer (POSM) production process.

The catalytic pyrolysis conditions further comprise a medium or large pore zeolite or molecular sieve. The medium or large pore zeolite or molecular sieve comprises beta zeolite (BEA structure), ferrierite (FER structure), mordenite (MOR structure), zeolite L (LTL structure), zeolite X (FAU structure), zeolite Y (FAU structure), ZSM-11 (MEL structure), ZSM-22 (TON structure), ZSM-23 (MTT structure), ZSM-5 (MFI structure), or a combination thereof.

In some embodiments, pyrolysis section 320 is configured to provide one or more pyrolysis reaction zones in series. In some embodiments wherein the one or more pyrolysis reaction zones in series, a reaction product of at least one pyrolysis reaction zone is separated into an intermediate product stream and a residual fraction, and at least one of the intermediate product stream and the residual fraction is fed to a downstream pyrolysis reaction zone.

In some embodiments, pyrolysis section 320 comprises one or more thermal pyrolysis zones. In some embodiments, pyrolysis section 320 comprises one or more catalytic pyrolysis zones. In some embodiments, pyrolysis section 320 comprises at least one thermal pyrolysis zone followed by at least one catalytic pyrolysis zone. In some embodiments, pyrolysis section 320 comprises at least one catalytic pyrolysis zone followed by at least one thermal pyrolysis zone. In some embodiments, pyrolysis section 320 comprises two or more pyrolysis zones in series (e.g., thermal pyrolysis/thermal pyrolysis, thermal pyrolysis/catalytic pyrolysis, catalytic pyrolysis/thermal pyrolysis, catalytic pyrolysis/catalytic pyrolysis). In some embodiments, pyrolysis section 320 comprises an upstream pyrolysis zone followed by a separation system followed by a downstream pyrolysis zone. The separation system separates the upstream pyrolysis effluent into a light stream and a heavy stream. The light stream is sent as feed to the downstream pyrolysis zone and the heavy stream is recycled to the upstream pyrolysis zone as additional feed.

The first intermediate product 321 is subjected to separation conditions in separation section 330 to produce second intermediate product 331 and first residual fraction 332. The second intermediate product 331 is subjected to hydrogenation conditions in hydrogenation section 340 to produce third intermediate product 341.

In some embodiments, the ratio of the styrene content of the first intermediate product 321 to the styrene content of the heavy aromatic waste stream 302 is greater than or equal to 1.01, greater than or equal to 1.02, greater than or equal to 1.05, greater than or equal to 1.10, greater than or equal to 1.15, or greater than or equal to 1.20.

In some embodiments, the second intermediate product 331 comprises ethylbenzene in an amount greater than or equal to 30 wt %, greater than or equal to 40 wt %, greater than or equal to 50 wt %, or greater than or equal to 60 wt %, based on the total weight of the second intermediate product.

In some embodiments, the third intermediate product 341 comprises ethylbenzene in an amount greater than or equal to 60 wt %, greater than or equal to 70 wt %, greater than or equal to 80 wt %, greater than or equal to 90 wt %, greater than or equal to 95 wt %, greater than or equal to 98 wt %, greater than or equal to 99 wt %, greater than or equal to 99.5 wt %, or greater than or equal to 99.9 wt %, based on the total weight of the third intermediate product.

The third intermediate product 341 is then fed to a POSM/SM process 390, wherein POSM/SM means the POSM process and/or the SM process. SM process means the direct dehydrogenation of EB to form styrene monomer. The third intermediate product 341 comprises EB. As part of POSM/SM process 390, EB is recovered from the third intermediate product 341, wherein the purity of the recovered EB is suitable for direct or indirect addition to the POSM and/or the SM process. Indirect addition means that one or more additional processing steps can be performed on the recovered stream to make it suitable for addition to the POSM and/or the SM process. The specific components in the heavy aromatic waste stream 302, the specific pyrolysis conditions in the pyrolysis section 320, the specific separation conditions in the separation section 330, and the specific hydrogenation conditions in the hydrogenation section 340 will result in different amounts of styrene in the third intermediate product 341. Examples of hydrogenation catalysts include any commercial hydrogenation catalysts known in the art such as NiMo, CoMo, Ni, Pd, Pt, Cu, and the like.

In some embodiments, ethylbenzene is recovered from the third intermediate product 341. In some embodiments, at least a portion of the EB is added to the POSM process, wherein EB is oxidized to form ethylbenzene hydroperoxide. The ethylbenzene hydroperoxide is then catalytically reacted the with propylene to form propylene oxide and 1-phenyl ethanol. The 1-phenyl ethanol is then dehydrated to produce styrene monomer. In some embodiments, at least a portion of the EB is added to the SM process, wherein EB is dehydrogenated to produce styrene monomer. In some embodiments, EB is added to both the POSM and SM processes.

In some embodiments, the first residual fraction 332 is added to the pyrolysis section 320 as additional feed via stream 333, sent outside the process for addition processing via stream 334, or a combination thereof.

FIG. 4 shows a process 400 to upgrade a heavy aromatic waste stream 402 into chemical feedstocks. The heavy aromatic waste stream 402 has a first styrene content. The process conditions and catalyst selection are optimized to achieve a desired product composition. In some embodiments, the process 400 comprises subjecting the heavy aromatic waste stream 402 to pyrolysis conditions in one or more pyrolysis reaction zones 420 to produce a first intermediate product 421 having a higher styrene content than the heavy aromatic waste stream and char 422.

The pyrolysis conditions and the one or more pyrolysis reaction zones 420 comprise thermal pyrolysis conditions in one or more thermal pyrolysis reaction zones, catalytic pyrolysis conditions in one or more catalytic pyrolysis zones, a combination thereof.

The thermal pyrolysis conditions comprise:
a) a temperature in the range of from about 200° C. to about 800° C., from 300° C. to 700° C., or from 400° C. to 600° C.;
b) a pressure in the range of from 5 kPa (absolute) to 450 kPa, from 13.3 kPa (absolute) to 400 kPa, from atmospheric pressure to 350 kPa, or from 100 kPa to 300 kPa; or
c) a combination thereof.

The catalytic pyrolysis conditions comprise:
a) a temperature in the range of from 300° C. to 600° C., from 250° C. to 450° C., from 280° C. to 420° C., or from 280° C. to 350° C.;
b) a pressure in the range of from 5 kPa (absolute) to 450 kPa, from 13.3 kPa (absolute) to 400 kPa, from atmospheric pressure to 350 kPa, or from 100 kPa to 300 kPa;
c) a weight hourly space velocity in the range of from 0.1 hr⁻¹ to 100 hr⁻¹, from 0.5 hr⁻¹ to 60 hr⁻¹, from 0.8 hr⁻¹ to 25 hr⁻¹, from 1.0 hr⁻¹ to 25 hr⁻¹, or from 3.0 hr⁻¹ to 25 hr⁻¹; or
d) a combination thereof.

The catalytic pyrolysis conditions further comprise a medium or large pore zeolite or molecular sieve. The medium or large pore zeolite or molecular sieve comprises beta zeolite (BEA structure), ferrierite (FER structure), mordenite (MOR structure), zeolite L (LTL structure), zeolite X (FAU structure), zeolite Y (FAU structure), ZSM-11 (MEL structure), ZSM-22 (TON structure), ZSM-23 (MTT structure), ZSM-5 (MFI structure), or a combination thereof.

In some embodiments, pyrolysis section 420 is configured to provide one or more pyrolysis reaction zones in series. In some embodiments wherein the one or more pyrolysis reaction zones in series, a reaction product of at least one pyrolysis reaction zone is separated into an intermediate product stream and a residual fraction, and at least one of the intermediate product stream and the residual fraction is fed to a downstream pyrolysis reaction zone.

In some embodiments, pyrolysis section 420 comprises one or more thermal pyrolysis zones. In some embodiments, pyrolysis section 420 comprises one or more catalytic pyrolysis zones. In some embodiments, pyrolysis section 420 comprises at least one thermal pyrolysis zone followed by at least one catalytic pyrolysis zone. In some embodiments, pyrolysis section 420 comprises at least one catalytic pyrolysis zone followed by at least one thermal pyrolysis zone. In some embodiments, pyrolysis section 420 comprises two or more pyrolysis zones in series (e.g., thermal pyrolysis/thermal pyrolysis, thermal pyrolysis/catalytic pyrolysis, catalytic pyrolysis/thermal pyrolysis, catalytic pyrolysis/catalytic pyrolysis). In some embodiments, pyrolysis section 420 comprises an upstream pyrolysis zone followed by a separation system followed by a downstream pyrolysis zone. The separation system separates the upstream pyrolysis effluent into a light stream and a heavy stream. The light stream is sent as feed to the downstream pyrolysis zone and the heavy stream is recycled to the upstream pyrolysis zone as additional feed.

The first intermediate product 421 is subjected to separation conditions in separation section 430 to produce third intermediate product 431 and first residual fraction 432. The second intermediate product 431 is subjected to hydrogenation conditions in hydrogenation section 440 to produce third intermediate product 441. Examples of hydrogenation catalysts include any commercial hydrogenation catalysts known in the art such as NiMo, CoMo, Ni, Pd, Pt, Cu, and the like.

In some embodiments, the ratio of the styrene content of the first intermediate product 421 to the styrene content of the heavy aromatic waste stream 402 is greater than or equal to 1.01, greater than or equal to 1.02, greater than or equal to 1.05, greater than or equal to 1.10, greater than or equal to 1.15, or greater than or equal to 1.20.

In some embodiments, the second intermediate product 431 comprises ethylbenzene in an amount greater than or equal to 30 wt %, greater than or equal to 40 wt %, greater than or equal to 50 wt %, or greater than or equal to 60 wt %, based on the total weight of the second intermediate product.

In some embodiments, the third intermediate product 441 comprises ethylbenzene in an amount greater than or equal to 60 wt %, greater than or equal to 70 wt %, greater than or equal to 80 wt %, greater than or equal to 90 wt %, greater than or equal to 95 wt %, greater than or equal to 98 wt %, greater than or equal to 99 wt %, greater than or equal to 99.5 wt %, or greater than or equal to 99.9 wt %, based on the total weight of the third intermediate product.

The third intermediate product 441 is then fed to a POSM/SM process 490, wherein POSM/SM means the POSM process and/or the SM process. SM process means the direct dehydrogenation of EB to form styrene monomer. The third intermediate product 441 comprises EB. As part of POSM/SM process 490, EB is recovered from the third intermediate product 441, wherein the purity of the recovered EB is suitable for direct or indirect addition to the POSM and/or the SM process. Indirect addition means that one or more additional processing steps can be performed on the recovered stream to make it suitable for addition to the POSM and/or the SM process. The specific components in the heavy aromatic waste stream 402, the specific pyrolysis conditions in the pyrolysis section 420, the specific hydrogenation conditions in the hydrogenation section 440, and the specific separation conditions in the separation section 430 will result in different amounts of styrene in the third intermediate product 441. Examples of hydrogenation catalysts include any commercial hydrogenation catalysts known in the art such as NiMo, CoMo, Ni, Pd, Pt, Cu, and the like.

In some embodiments, ethylbenzene is recovered from the third intermediate product 441. In some embodiments, at least a portion of the EB is added to the POSM process, wherein EB is oxidized to form ethylbenzene hydroperoxide. The ethylbenzene hydroperoxide is then catalytically reacted the with propylene to form propylene oxide and 1-phenyl ethanol. The 1-phenyl ethanol is then dehydrated to produce styrene monomer. In some embodiments, at least a portion of the EB is added to the SM process, wherein EB is dehydrogenated to produce styrene monomer. In some embodiments, EB is added to both the POSM and SM processes.

In some embodiments, the first residual fraction 432 is added to a second the pyrolysis section 425 as feed via stream 434, added to a first the pyrolysis section 425 as additional feed via stream 433, or a combination thereof. First residual fraction 434 is subjected to pyrolysis conditions in one or more pyrolysis reaction zones 425 to produce a fourth intermediate product 426 having a higher styrene content than the first residual fraction 432 and char 427.

The pyrolysis conditions and the one or more pyrolysis reaction zones 425 comprise thermal pyrolysis conditions in one or more thermal pyrolysis reaction zones, catalytic pyrolysis conditions in one or more catalytic pyrolysis zones, a combination thereof.

The thermal pyrolysis conditions comprise:
a) a temperature in the range of from about 200° C. to about 800° C., from 300° C. to 700° C., or from 400° C. to 600° C.;
b) a pressure in the range of from 5 kPa (absolute) to 450 kPa, from 13.3 kPa (absolute) to 400 kPa, from atmospheric pressure to 350 kPa, or from 100 kPa to 300 kPa; or
c) a combination thereof.

The catalytic pyrolysis conditions comprise:
a) a temperature in the range of from 300° C. to 600° C., from 250° C. to 450° C., from 280° C. to 420° C., or from 280° C. to 350° C.;
b) a pressure in the range of from 5 kPa (absolute) to 450 kPa, from 13.3 kPa (absolute) to 400 kPa, from atmospheric pressure to 350 kPa, or from 100 kPa to 300 kPa;
c) a weight hourly space velocity in the range of from 0.1 hr⁻¹ to 100 hr⁻¹, from 0.5 hr⁻¹ to 60 hr⁻¹, from 0.8 hr⁻¹ to 25 hr⁻¹, from 1.0 hr⁻¹ to 25 hr⁻¹, or from 3.0 hr⁻¹ to 25 hr⁻¹; or
d) a combination thereof.

The catalytic pyrolysis conditions further comprise a medium or large pore zeolite or molecular sieve. The medium or large pore zeolite or molecular sieve comprises beta zeolite (BEA structure), ferrierite (FER structure), mordenite (MOR structure), zeolite L (LTL structure), zeolite X (FAU structure), zeolite Y (FAU structure), ZSM-11 (MEL structure), ZSM-22 (TON structure), ZSM-23 (MTT structure), ZSM-5 (MFI structure), or a combination thereof.

In some embodiments, pyrolysis section 425 is configured to provide one or more pyrolysis reaction zones in series. In some embodiments wherein the one or more pyrolysis reaction zones in series, a reaction product of at least one pyrolysis reaction zone is separated into an intermediate product stream and a residual fraction, and at least one of the intermediate product stream and the residual fraction is fed to a downstream pyrolysis reaction zone.

In some embodiments, pyrolysis section 425 comprises one or more thermal pyrolysis zones. In some embodiments, pyrolysis section 425 comprises one or more catalytic pyrolysis zones. In some embodiments, pyrolysis section 425 comprises at least one thermal pyrolysis zone followed by at least one catalytic pyrolysis zone. In some embodiments, pyrolysis section 425 comprises at least one catalytic pyrolysis zone followed by at least one thermal pyrolysis zone. In some embodiments, pyrolysis section 425 comprises two or more pyrolysis zones in series (e.g., thermal pyrolysis/thermal pyrolysis, thermal pyrolysis/catalytic pyrolysis, catalytic pyrolysis/thermal pyrolysis, catalytic pyrolysis/catalytic pyrolysis). In some embodiments, pyrolysis section 425 comprises an upstream pyrolysis zone followed by a separation system followed by a downstream pyrolysis zone. The separation system separates the upstream pyrolysis effluent into a light stream and a heavy stream. The light stream is sent as feed to the downstream pyrolysis zone and the heavy stream is recycled to the upstream pyrolysis zone as additional feed.

The fourth intermediate product 421 is subjected to separation conditions in separation section 435 to produce fifth intermediate product 436 and second residual fraction 437.

In some embodiments, the ratio of the styrene content of the fourth intermediate product 426 to the styrene content of the first residual fraction 434 is greater than or equal to 1.01, greater than or equal to 1.02, greater than or equal to 1.05, greater than or equal to 1.10, greater than or equal to 1.15, or greater than or equal to 1.20.

In some embodiments, the fifth intermediate product 436 comprises ethylbenzene in an amount greater than or equal to 30 wt %, greater than or equal to 40 wt %, greater than or equal to 50 wt %, or greater than or equal to 60 wt %, based on the total weight of the second intermediate product.

The fifth intermediate product 436 is then fed to a POSM/SM process 490, wherein POSM/SM means the POSM process and/or the SM process. SM process means the direct dehydrogenation of EB to form styrene monomer. The fifth intermediate product 436 comprises one or more of styrene, benzene, ethylene, and propylene. As part of POSM/SM process 490, one or more of styrene, benzene, ethylene, and propylene is recovered from the fifth intermediate product 436, wherein the purity of the recovered styrene, benzene, ethylene, and propylene is suitable for direct or indirect addition to the POSM and/or the SM process. Indirect addition means that one or more additional processing steps can be performed on the recovered stream to make it suitable for addition to the POSM and/or the SM process. The specific components in the first residual fraction 434, the specific pyrolysis conditions in the pyrolysis reaction zone 425, and the specific separation conditions in the separation section 435 will result in different amounts of styrene, benzene, ethylene, and/or propylene in the fifth intermediate product 436.

In some embodiments, styrene is recovered from the fifth intermediate product 436. The styrene is hydrogenated to form EB. Examples of hydrogenation catalysts include any commercial hydrogenation catalysts known in the art such as NiMo, CoMo, Ni, Pd, Pt, Cu, and the like. In some embodiments, at least a portion of the EB is added to the POSM process, wherein EB is oxidized to form ethylbenzene hydroperoxide. The ethylbenzene hydroperoxide is then catalytically reacted the with propylene to form propylene oxide and 1-phenyl ethanol. The 1-phenyl ethanol is then dehydrated to produce styrene monomer. In some embodiments, at least a portion of the EB is added to the SM process, wherein EB is dehydrogenated to produce styrene monomer. In some embodiments, EB is added to both the POSM and SM processes.

In some embodiments, benzene is recovered from the fifth intermediate product 436. The benzene is reacted with ethylene to form EB. In some embodiments, at least a portion of the EB is added to the POSM process, wherein EB is oxidized to form ethylbenzene hydroperoxide. The ethylbenzene hydroperoxide is then catalytically reacted the with propylene to form propylene oxide and 1-phenyl ethanol. The 1-phenyl ethanol is then dehydrated to produce styrene monomer. In some embodiments, at least a portion of the EB is added to the SM process, wherein EB is dehydrogenated to produce styrene monomer. In some embodiments, EB is added to both the POSM and SM processes.

In some embodiments, ethylene is recovered from the fifth intermediate product 436. The ethylene is reacted with benzene to form EB. In some embodiments, at least a portion of the EB is added to the POSM process, wherein EB is oxidized to form ethylbenzene hydroperoxide. The ethylbenzene hydroperoxide is then catalytically reacted the with propylene to form propylene oxide and 1-phenyl ethanol. The 1-phenyl ethanol is then dehydrated to produce styrene monomer. In some embodiments, at least a portion of the EB is added to the SM process, wherein EB is dehydrogenated to produce styrene monomer. In some embodiments, EB is added to both the POSM and SM processes.

In some embodiments, propylene is recovered from the fifth intermediate product 436. The propylene is added to the POSM process, wherein it is reacted with ethylbenzene hydroperoxide to form propylene oxide and 1-phenyl ethanol. The 1-phenyl ethanol is then dehydrated to produce styrene monomer.

In some embodiments, the second residual fraction 437 is added to the pyrolysis section 420 as additional feed via streams 438, 438b, added to the pyrolysis section 425 as additional feed via streams 438, 438a, sent outside the process for addition processing via stream 439, or a combination thereof.

FIG. 5 shows a process 500 to upgrade a heavy aromatic waste stream 502 into chemical feedstocks. The heavy aromatic waste stream 502 has a first styrene content. The process conditions and catalyst selection are optimized to achieve a desired product composition. In some embodiments, the process 500 comprises subjecting the heavy aromatic waste stream 502 to pyrolysis conditions in one or more pyrolysis reaction zones 520 to produce a first intermediate product 521 having a higher styrene content than the heavy aromatic waste stream and char 522.

The pyrolysis conditions and the one or more pyrolysis reaction zones 520 comprise thermal pyrolysis conditions in one or more thermal pyrolysis reaction zones, catalytic pyrolysis conditions in one or more catalytic pyrolysis zones, a combination thereof.

The thermal pyrolysis conditions comprise:
a) a temperature in the range of from about 200° C. to about 800° C., from 300° C. to 700° C., or from 400° C. to 600° C.;
b) a pressure in the range of from 5 kPa (absolute) to 450 kPa, from 13.3 kPa (absolute) to 400 kPa, from atmospheric pressure to 350 kPa, or from 100 kPa to 300 kPa; or
c) a combination thereof.

The catalytic pyrolysis conditions comprise:
a) a temperature in the range of from 300° C. to 600° C., from 250° C. to 450° C., from 280° C. to 420° C., or from 280° C. to 350° C.;
b) a pressure in the range of from 5 kPa (absolute) to 450 kPa, from 13.3 kPa (absolute) to 400 kPa, from atmospheric pressure to 350 kPa, or from 100 kPa to 300 kPa;
c) a weight hourly space velocity in the range of from 0.1 hr⁻¹ to 100 hr⁻¹, from 0.5 hr⁻¹ to 60 hr⁻¹, from 0.8 hr⁻¹ to 25 hr⁻¹, from 1.0 hr⁻¹ to 25 hr⁻¹, or from 3.0 hr⁻¹ to 25 hr⁻¹; or
d) a combination thereof.

In some embodiments, catalytic cracking the fuel oil occurs at a temperature greater than about 380° C. In some embodiments, catalytic cracking the fuel oil occurs at a temperature greater than about 480° C. In some embodiments, catalytic cracking the fuel oil occurs at a temperature greater than about 580° C. In some embodiments, catalytic cracking the fuel oil occurs at a temperature in the range of from about 350° C. to about 650° C. In some embodiments, catalytic cracking the fuel oil occurs at a temperature in the range of from about 350° C. to about 450° C. In some embodiments, catalytic cracking the fuel oil occurs at a temperature in the range of from about 450° C. to about 550° C. In some embodiments, catalytic cracking the fuel oil occurs at a temperature in the range of from about 550° C. to about 650° C. In some embodiments, the catalyst is a zeolite. In some embodiments, the catalyst is a H-ZSM-5 zeolite. In some embodiments, the fuel oil is obtained from a propylene oxide/styrene monomer (POSM) production process.

The catalytic pyrolysis conditions further comprise a medium or large pore zeolite or molecular sieve. The medium or large pore zeolite or molecular sieve comprises beta zeolite (BEA structure), ferrierite (FER structure), mordenite (MOR structure), zeolite L (LTL structure), zeolite X (FAU structure), zeolite Y (FAU structure), ZSM-11 (MEL structure), ZSM-22 (TON structure), ZSM-23 (MTT structure), ZSM-5 (MFI structure), or a combination thereof.

In some embodiments, pyrolysis section 520 is configured to provide one or more pyrolysis reaction zones in series. In some embodiments wherein the one or more pyrolysis reaction zones in series, a reaction product of at least one pyrolysis reaction zone is separated into an intermediate product stream and a residual fraction, and at least one of the intermediate product stream and the residual fraction is fed to a downstream pyrolysis reaction zone.

In some embodiments, pyrolysis section 520 comprises one or more thermal pyrolysis zones. In some embodiments, pyrolysis section 520 comprises one or more catalytic pyrolysis zones. In some embodiments, pyrolysis section 520 comprises at least one thermal pyrolysis zone followed by at least one catalytic pyrolysis zone. In some embodiments, pyrolysis section 520 comprises at least one catalytic pyrolysis zone followed by at least one thermal pyrolysis zone. In some embodiments, pyrolysis section 520 comprises two or more pyrolysis zones in series (e.g., thermal pyrolysis/thermal pyrolysis, thermal pyrolysis/catalytic pyrolysis, catalytic pyrolysis/thermal pyrolysis, catalytic pyrolysis/catalytic pyrolysis). In some embodiments, pyrolysis section 520 comprises an upstream pyrolysis zone followed by a separation system followed by a downstream pyrolysis zone. The separation system separates the upstream pyrolysis effluent into a light stream and a heavy stream. The light stream is sent as feed to the downstream pyrolysis zone and the heavy stream is recycled to the upstream pyrolysis zone as additional feed.

The first intermediate product 521 is subjected to separation conditions in separation section 530 to produce second intermediate product 531 and first residual fraction 532. The second intermediate product 531 is subjected to hydrogenation conditions in hydrogenation section 540 to produce third intermediate product 541. Examples of hydrogenation catalysts include any commercial hydrogenation catalysts known in the art such as NiMo, CoMo, Ni, Pd, Pt, Cu, and the like. The third intermediate product 541 is subjected to separation conditions in separation system 550 to produce fourth intermediate product 551.

In some embodiments, the ratio of the styrene content of the first intermediate product 521 to the styrene content of the heavy aromatic waste stream 502 is greater than or equal to 1.01, greater than or equal to 1.02, greater than or equal to 1.05, greater than or equal to 1.10, greater than or equal to 1.15, or greater than or equal to 1.20.

In some embodiments, the second intermediate product 531 comprises ethylbenzene in an amount greater than or equal to 30 wt %, greater than or equal to 40 wt %, greater than or equal to 50 wt %, or greater than or equal to 60 wt %, based on the total weight of the second intermediate product.

In some embodiments, the third intermediate product 541 comprises ethylbenzene in an amount greater than or equal to 60 wt %, greater than or equal to 70 wt %, greater than or equal to 80 wt %, greater than or equal to 90 wt %, greater than or equal to 95 wt %, greater than or equal to 98 wt %, greater than or equal to 99 wt %, greater than or equal to 99.5 wt %, or greater than or equal to 99.9 wt %, based on the total weight of the third intermediate product.

The third intermediate product 541 is subjected to separation conditions in separation section 550 to produce fourth intermediate product 551 and second residual fraction 552. The fourth intermediate product 551 is then fed to a POSM/SM process 590, wherein POSM/SM means the POSM process and/or the SM process. SM process means the direct dehydrogenation of EB to form styrene monomer. The fourth intermediate product 551 comprises EB. As part of POSM/SM process 590, EB is recovered from the fourth intermediate product 551, wherein the purity of the recovered EB is suitable for direct or indirect addition to the POSM and/or the SM process. Indirect addition means that one or more additional processing steps can be performed on the recovered stream to make it suitable for addition to the POSM and/or the SM process. The specific components in the heavy aromatic waste stream 502, the specific pyrolysis conditions in the pyrolysis section 520, the specific separation conditions in the separation sections 530 and 550, and the specific hydrogenation conditions in the hydrogenation section 540 will result in different amounts of styrene in the fourth intermediate product 551. Examples of hydrogenation catalysts include any commercial hydrogenation catalysts known in the art such as NiMo, CoMo, Ni, Pd, Pt, Cu, and the like.

In some embodiments, ethylbenzene is recovered from the fourth intermediate product 551. In some embodiments, at least a portion of the EB is added to the POSM process, wherein EB is oxidized to form ethylbenzene hydroperoxide. The ethylbenzene hydroperoxide is then catalytically reacted the with propylene to form propylene oxide and 1-phenyl ethanol. The 1-phenyl ethanol is then dehydrated to produce styrene monomer. In some embodiments, at least a portion of the EB is added to the SM process, wherein EB is dehydrogenated to produce styrene monomer. In some embodiments, EB is added to both the POSM and SM processes.

In some embodiments, the first residual fraction 532 and/or the second residual fraction 552 are added to the pyrolysis section 520 as additional feed via stream 533, sent outside the process for addition processing via stream 434 and/or 554, or a combination thereof.

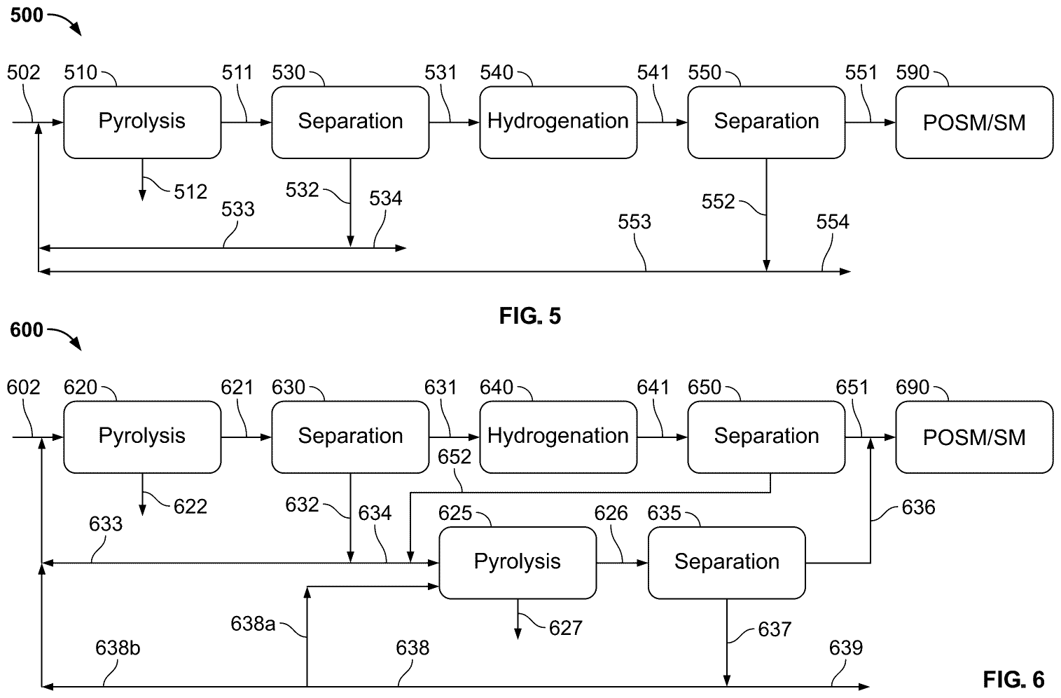

FIG. 6 shows a process 600 to upgrade a heavy aromatic waste stream 602 into chemical feedstocks. The heavy aromatic waste stream 602 has a first styrene content. The process conditions and catalyst selection are optimized to achieve a desired product composition. In some embodiments, the process 600 comprises subjecting the heavy aromatic waste stream 602 to pyrolysis conditions in one or more pyrolysis reaction zones 620 to produce a first intermediate product 621 having a higher styrene content than the heavy aromatic waste stream and char 622.

The pyrolysis conditions and the one or more pyrolysis reaction zones 620 comprise thermal pyrolysis conditions in one or more thermal pyrolysis reaction zones, catalytic pyrolysis conditions in one or more catalytic pyrolysis zones, a combination thereof.

The thermal pyrolysis conditions comprise:
a) a temperature in the range of from about 200° C. to about 800° C., from 300° C. to 700° C., or from 400° C. to 600° C.;
b) a pressure in the range of from 5 kPa (absolute) to 450 kPa, from 13.3 kPa (absolute) to 400 kPa, from atmospheric pressure to 350 kPa, or from 100 kPa to 300 kPa; or
c) a combination thereof.

The catalytic pyrolysis conditions comprise:
a) a temperature in the range of from 300° C. to 600° C., from 250° C. to 450° C., from 280° C. to 420° C., or from 280° C. to 350° C.;
b) a pressure in the range of from 5 kPa (absolute) to 450 kPa, from 13.3 kPa (absolute) to 400 kPa, from atmospheric pressure to 350 kPa, or from 100 kPa to 300 kPa;
c) a weight hourly space velocity in the range of from 0.1 hr⁻¹ to 100 hr⁻¹, from 0.5 hr⁻¹ to 60 hr⁻¹, from 0.8 hr⁻¹ to 25 hr⁻¹, from 1.0 hr⁻¹ to 25 hr⁻¹, or from 3.0 hr⁻¹ to 25 hr⁻¹; or
d) a combination thereof.

In some embodiments, catalytic cracking the fuel oil occurs at a temperature greater than about 380° C. In some embodiments, catalytic cracking the fuel oil occurs at a temperature greater than about 480° C. In some embodiments, catalytic cracking the fuel oil occurs at a temperature greater than about 580° C. In some embodiments, catalytic cracking the fuel oil occurs at a temperature in the range of from about 350° C. to about 650° C. In some embodiments, catalytic cracking the fuel oil occurs at a temperature in the range of from about 350° C. to about 450° C. In some embodiments, catalytic cracking the fuel oil occurs at a temperature in the range of from about 450° C. to about 550° C. In some embodiments, catalytic cracking the fuel oil occurs at a temperature in the range of from about 550° C. to about 650° C. In some embodiments, the catalyst is a zeolite. In some embodiments, the catalyst is a H-ZSM-5 zeolite. In some embodiments, the fuel oil is obtained from a propylene oxide/styrene monomer (POSM) production process.

The catalytic pyrolysis conditions further comprise a medium or large pore zeolite or molecular sieve. The medium or large pore zeolite or molecular sieve comprises beta zeolite (BEA structure), ferrierite (FER structure), mordenite (MOR structure), zeolite L (LTL structure), zeolite X (FAU structure), zeolite Y (FAU structure), ZSM-11 (MEL structure), ZSM-22 (TON structure), ZSM-23 (MTT structure), ZSM-5 (MFI structure), or a combination thereof.

In some embodiments, pyrolysis section 620 is configured to provide one or more pyrolysis reaction zones in series. In some embodiments wherein the one or more pyrolysis reaction zones in series, a reaction product of at least one pyrolysis reaction zone is separated into an intermediate product stream and a residual fraction, and at least one of the intermediate product stream and the residual fraction is fed to a downstream pyrolysis reaction zone.

In some embodiments, pyrolysis section 620 comprises one or more thermal pyrolysis zones. In some embodiments, pyrolysis section 620 comprises one or more catalytic pyrolysis zones. In some embodiments, pyrolysis section 620 comprises at least one thermal pyrolysis zone followed by at least one catalytic pyrolysis zone. In some embodiments, pyrolysis section 620 comprises at least one catalytic pyrolysis zone followed by at least one thermal pyrolysis zone. In some embodiments, pyrolysis section 620 comprises two or more pyrolysis zones in series (e.g., thermal pyrolysis/thermal pyrolysis, thermal pyrolysis/catalytic pyrolysis, catalytic pyrolysis/thermal pyrolysis, catalytic pyrolysis/catalytic pyrolysis). In some embodiments, pyrolysis section 620 comprises an upstream pyrolysis zone followed by a separation system followed by a downstream pyrolysis zone. The separation system separates the upstream pyrolysis effluent into a light stream and a heavy stream. The light stream is sent as feed to the downstream pyrolysis zone and the heavy stream is recycled to the upstream pyrolysis zone as additional feed.

The first intermediate product 621 is subjected to separation conditions in separation section 630 to produce third intermediate product 631 and first residual fraction 632. The second intermediate product 631 is subjected to hydrogenation conditions in hydrogenation section 640 to produce third intermediate product 641. Examples of hydrogenation catalysts include any commercial hydrogenation catalysts known in the art such as NiMo, CoMo, Ni, Pd, Pt, Cu, and the like. The third intermediate product 641 is subjected to separation conditions in separation system 550 to produce fourth intermediate product 651

In some embodiments, the ratio of the styrene content of the first intermediate product 621 to the styrene content of the heavy aromatic waste stream 602 is greater than or equal to 1.01, greater than or equal to 1.02, greater than or equal to 1.05, greater than or equal to 1.10, greater than or equal to 1.15, or greater than or equal to 1.20.

In some embodiments, the second intermediate product 631 comprises ethylbenzene in an amount greater than or equal to 30 wt %, greater than or equal to 40 wt %, greater than or equal to 50 wt %, or greater than or equal to 60 wt %, based on the total weight of the second intermediate product.

In some embodiments, the third intermediate product 641 comprises ethylbenzene in an amount greater than or equal to 60 wt %, greater than or equal to 70 wt %, greater than or equal to 80 wt %, greater than or equal to 90 wt %, greater than or equal to 95 wt %, greater than or equal to 98 wt %, greater than or equal to 99 wt %, greater than or equal to 99.5 wt %, or greater than or equal to 99.9 wt %, based on the total weight of the third intermediate product.

The third intermediate product 641 is subjected to separation conditions in separation section 550 to produce fourth intermediate product 551 and second residual fraction 652. The fourth intermediate product 551 is then fed to a POSM/SM process 690, wherein POSM/SM means the POSM process and/or the SM process. SM process means the direct dehydrogenation of EB to form styrene monomer. The fourth intermediate product 651 comprises EB. As part of POSM/SM process 690, EB is recovered from the fourth intermediate product 651, wherein the purity of the recovered EB is suitable for direct or indirect addition to the POSM and/or the SM process. Indirect addition means that one or more additional processing steps can be performed on the recovered stream to make it suitable for addition to the POSM and/or the SM process. The specific components in the heavy aromatic waste stream 602, the specific pyrolysis conditions in the pyrolysis section 620, the specific separation conditions in the separation sections 630 and 650, and the specific hydrogenation conditions in the hydrogenation section 640 will result in different amounts of styrene in the fourth intermediate product 651. Examples of hydrogenation catalysts include any commercial hydrogenation catalysts known in the art such as NiMo, CoMo, Ni, Pd, Pt, Cu, and the like.

In some embodiments, ethylbenzene is recovered from the fourth intermediate product 651. In some embodiments, at least a portion of the EB is added to the POSM process, wherein EB is oxidized to form ethylbenzene hydroperoxide. The ethylbenzene hydroperoxide is then catalytically reacted the with propylene to form propylene oxide and 1-phenyl ethanol. The 1-phenyl ethanol is then dehydrated to produce styrene monomer. In some embodiments, at least a portion of the EB is added to the SM process, wherein EB is dehydrogenated to produce styrene monomer. In some embodiments, EB is added to both the POSM and SM processes.

In some embodiments, the first residual fraction 632 and/or the second residual fraction 652 are added to a second the pyrolysis section 625 as feed via stream 634, added to a first the pyrolysis section 625 as additional feed via stream 633, or a combination thereof. First residual fraction 634 is subjected to pyrolysis conditions in one or more pyrolysis reaction zones 625 to produce a fifth intermediate product 626 having a higher styrene content than the first residual fraction 632 and char 627.

The pyrolysis conditions and the one or more pyrolysis reaction zones 625 comprise thermal pyrolysis conditions in one or more thermal pyrolysis reaction zones, catalytic pyrolysis conditions in one or more catalytic pyrolysis zones, a combination thereof.

The thermal pyrolysis conditions comprise:
a) a temperature in the range of from about 200° C. to about 800° C., from 300° C. to 700° C., or from 400° C. to 600° C.;
b) a pressure in the range of from 5 kPa (absolute) to 450 kPa, from 13.3 kPa (absolute) to 400 kPa, from atmospheric pressure to 350 kPa, or from 100 kPa to 300 kPa; or
c) a combination thereof.

The catalytic pyrolysis conditions comprise:
a) a temperature in the range of from 300° C. to 600° C., from 250° C. to 450° C., from 280° C. to 420° C., or from 280° C. to 350° C.;
b) a pressure in the range of from 5 kPa (absolute) to 450 kPa, from 13.3 kPa (absolute) to 400 kPa, from atmospheric pressure to 350 kPa, or from 100 kPa to 300 kPa;
c) a weight hourly space velocity in the range of from 0.1 hr⁻¹ to 100 hr⁻¹, from 0.5 hr⁻¹ to 60 hr⁻¹, from 0.8 hr⁻¹ to 25 hr⁻¹, from 1.0 hr⁻¹ to 25 hr⁻¹, or from 3.0 hr⁻¹ to 25 hr⁻¹; or
d) a combination thereof.

In some embodiments, catalytic cracking the fuel oil occurs at a temperature greater than about 380° C. In some embodiments, catalytic cracking the fuel oil occurs at a temperature greater than about 480° C. In some embodiments, catalytic cracking the fuel oil occurs at a temperature greater than about 580° C. In some embodiments, catalytic cracking the fuel oil occurs at a temperature in the range of from about 350° C. to about 650° C. In some embodiments, catalytic cracking the fuel oil occurs at a temperature in the range of from about 350° C. to about 450° C. In some embodiments, catalytic cracking the fuel oil occurs at a temperature in the range of from about 450° C. to about 550° C. In some embodiments, catalytic cracking the fuel oil occurs at a temperature in the range of from about 550° C. to about 650° C. In some embodiments, the catalyst is a zeolite. In some embodiments, the catalyst is a H-ZSM-5 zeolite. In some embodiments, the fuel oil is obtained from a propylene oxide/styrene monomer (POSM) production process.

The catalytic pyrolysis conditions further comprise a medium or large pore zeolite or molecular sieve. The medium or large pore zeolite or molecular sieve comprises beta zeolite (BEA structure), ferrierite (FER structure), mordenite (MOR structure), zeolite L (LTL structure), zeolite X (FAU structure), zeolite Y (FAU structure), ZSM-11 (MEL structure), ZSM-22 (TON structure), ZSM-23 (MTT structure), ZSM-5 (MFI structure), or a combination thereof.

In some embodiments, pyrolysis section 625 is configured to provide one or more pyrolysis reaction zones in series. In some embodiments wherein the one or more pyrolysis reaction zones in series, a reaction product of at least one pyrolysis reaction zone is separated into an intermediate product stream and a residual fraction, and at least one of the intermediate product stream and the residual fraction is fed to a downstream pyrolysis reaction zone.

In some embodiments, pyrolysis section 625 comprises one or more thermal pyrolysis zones. In some embodiments, pyrolysis section 625 comprises one or more catalytic pyrolysis zones. In some embodiments, pyrolysis section 625 comprises at least one thermal pyrolysis zone followed by at least one catalytic pyrolysis zone. In some embodiments, pyrolysis section 625 comprises at least one catalytic pyrolysis zone followed by at least one thermal pyrolysis zone. In some embodiments, pyrolysis section 625 comprises two or more pyrolysis zones in series (e.g., thermal pyrolysis/thermal pyrolysis, thermal pyrolysis/catalytic pyrolysis, catalytic pyrolysis/thermal pyrolysis, catalytic pyrolysis/catalytic pyrolysis). In some embodiments, pyrolysis section 625 comprises an upstream pyrolysis zone followed by a separation system followed by a downstream pyrolysis zone. The separation system separates the upstream pyrolysis effluent into a light stream and a heavy stream. The light stream is sent as feed to the downstream pyrolysis zone and the heavy stream is recycled to the upstream pyrolysis zone as additional feed.

The fifth intermediate product 626 is subjected to separation conditions in separation section 635 to produce sixth intermediate product 636 and third residual fraction 637.

In some embodiments, the ratio of the styrene content of the fifth intermediate product 626 to the styrene content of the first residual fraction 634 and/or the second residual fraction 652 is greater than or equal to 1.01, greater than or equal to 1.02, greater than or equal to 1.05, greater than or equal to 1.10, greater than or equal to 1.15, or greater than or equal to 1.20.

In some embodiments, the sixth intermediate product 636 comprises ethylbenzene in an amount greater than or equal to 30 wt %, greater than or equal to 40 wt %, greater than or equal to 50 wt %, or greater than or equal to 60 wt %, based on the total weight of the second intermediate product.

The sixth intermediate product 636 is then fed to a POSM/SM process 690, wherein POSM/SM means the POSM process and/or the SM process. SM process means the direct dehydrogenation of EB to form styrene monomer. The sixth intermediate product 636 comprises one or more of styrene, benzene, ethylene, and propylene. As part of POSM/SM process 690, one or more of styrene, benzene, ethylene, and propylene is recovered from the sixth intermediate product 636, wherein the purity of the recovered styrene, benzene, ethylene, and propylene is suitable for direct or indirect addition to the POSM and/or the SM process. Indirect addition means that one or more additional processing steps can be performed on the recovered stream to make it suitable for addition to the POSM and/or the SM process. The specific components in the first residual fraction 634 and/or the second residual fraction 652, the specific pyrolysis conditions in the pyrolysis reaction zone 625, and the specific separation conditions in the separation section 635 will result in different amounts of styrene, benzene, ethylene, and/or propylene in the sixth intermediate product 636.

In some embodiments, styrene is recovered from the sixth intermediate product 636. The styrene is hydrogenated to form EB. Examples of hydrogenation catalysts include any commercial hydrogenation catalysts known in the art such as NiMo, CoMo, Ni, Pd, Pt, Cu, and the like. In some embodiments, at least a portion of the EB is added to the POSM process, wherein EB is oxidized to form ethylbenzene hydroperoxide. The ethylbenzene hydroperoxide is then catalytically reacted the with propylene to form propylene oxide and 1-phenyl ethanol. The 1-phenyl ethanol is then dehydrated to produce styrene monomer. In some embodiments, at least a portion of the EB is added to the SM process, wherein EB is dehydrogenated to produce styrene monomer. In some embodiments, EB is added to both the POSM and SM processes.

In some embodiments, benzene is recovered from the sixth intermediate product 636. The benzene is reacted with ethylene to form EB. In some embodiments, at least a portion of the EB is added to the POSM process, wherein EB is oxidized to form ethylbenzene hydroperoxide. The ethylbenzene hydroperoxide is then catalytically reacted the with propylene to form propylene oxide and 1-phenyl ethanol. The 1-phenyl ethanol is then dehydrated to produce styrene monomer. In some embodiments, at least a portion of the EB is added to the SM process, wherein EB is dehydrogenated to produce styrene monomer. In some embodiments, EB is added to both the POSM and SM processes.

In some embodiments, ethylene is recovered from the sixth intermediate product 636. The ethylene is reacted with benzene to form EB. In some embodiments, at least a portion of the EB is added to the POSM process, wherein EB is oxidized to form ethylbenzene hydroperoxide. The ethylbenzene hydroperoxide is then catalytically reacted the with propylene to form propylene oxide and 1-phenyl ethanol. The 1-phenyl ethanol is then dehydrated to produce styrene monomer. In some embodiments, at least a portion of the EB is added to the SM process, wherein EB is dehydrogenated to produce styrene monomer. In some embodiments, EB is added to both the POSM and SM processes.

In some embodiments, propylene is recovered from the sixth intermediate product 636. The propylene is added to the POSM process, wherein it is reacted with ethylbenzene hydroperoxide to form propylene oxide and 1-phenyl ethanol. The 1-phenyl ethanol is then dehydrated to produce styrene monomer.

In some embodiments, the third residual fraction 637 is added to the pyrolysis section 620 as additional feed via streams 638, 638b, added to the pyrolysis section 625 as additional feed via streams 638, 638a, sent outside the process for addition processing via stream 639, or a combination thereof.

### Certain Embodiments

Embodiment A1. A process to upgrade a heavy aromatic waste stream to chemical feedstocks, the process comprising:
a) subjecting the heavy aromatic waste stream to pyrolysis conditions in one or more first pyrolysis reaction zones to produce a first intermediate product having a higher styrene content than the heavy aromatic waste stream;
b) subjecting the first intermediate product to hydrogenation conditions in a hydrogenation reaction zone to produce a second intermediate product comprising ethylbenzene; and
c) subjecting the second intermediate product to separation conditions to produce a third intermediate product comprising ethylbenzene and a first residual fraction.

Embodiment A2. The process of Embodiment A1, wherein the first pyrolysis conditions and the one or more first pyrolysis reaction zones comprise:
a) thermal pyrolysis conditions in one or more thermal pyrolysis reaction zones;
b) catalytic pyrolysis conditions in one or more catalytic pyrolysis zones; or
c) a combination thereof.

Embodiment A3. The process of Embodiment A2, wherein the thermal pyrolysis conditions comprise:
a) a temperature in the range of from about 200° C. to about 800° C., from 300° C. to 700° C., or from 400° C. to 600° C.;
b) a pressure in the range of from 5 kPa (absolute) to 450 kPa, from 13.3 kPa (absolute) to 400 kPa, from atmospheric pressure to 350 kPa, or from 100 kPa to 300 kPa; or
c) a combination thereof.

Embodiment A4. The process any one of Embodiments A2-A3, wherein the catalytic pyrolysis conditions comprise:
a) a temperature in the range of from 300° C. to 600° C., from 250° C. to 450° C., from 280° C. to 420° C., or from 280° C. to 350° C.;
b) a pressure in the range of from 5 kPa (absolute) to 450 kPa, from 13.3 kPa (absolute) to 400 kPa, from atmospheric pressure to 350 kPa, or from 100 kPa to 300 kPa;
c) a weight hourly space velocity in the range of from 0.1 hr⁻¹ to 100 hr⁻¹, from 0.5 hr⁻¹ to 60 hr⁻¹, from 0.8 hr⁻¹ to 25 hr⁻¹, from 1.0 hr⁻¹ to 25 hr⁻¹, or from 3.0 hr⁻¹ to 25 hr⁻¹; or
d) a combination thereof.

Embodiment A5. The process any one of Embodiments A2-A4, wherein the one or more catalytic pyrolysis zones each independently comprise a medium or large pore zeolite or molecular sieve.

Embodiment A6. The process of Embodiment A5, wherein the medium or large pore zeolite or molecular sieve comprises beta zeolite (BEA structure), ferrierite (FER structure), mordenite (MOR structure), zeolite L (LTL structure), zeolite X (FAU structure), zeolite Y (FAU structure), ZSM-11 (MEL structure), ZSM-22 (TON structure), ZSM-23 (MTT structure), ZSM-5 (MFI structure), or a combination thereof.

Embodiment A7. The process any one of Embodiments A1-A6, wherein the ratio of the styrene content of the first intermediate product to the styrene content of the heavy aromatic waste stream is greater than or equal to 1.01, greater than or equal to 1.02, greater than or equal to 1.05, greater than or equal to 1.10, greater than or equal to 1.15, or greater than or equal to 1.20.

Embodiment A8. The process any one of Embodiments A1-!7, wherein the second intermediate product comprises ethylbenzene in an amount greater than or equal to 30 wt %, greater than or equal to 40 wt %, greater than or equal to 50 wt %, or greater than or equal to 60 wt %, based on the total weight of the second intermediate product.

Embodiment A9. The process any one of Embodiments A1-A8, wherein the third intermediate product comprises ethylbenzene in an amount greater than or equal to 60 wt %, greater than or equal to 70 wt %, greater than or equal to 80 wt %, greater than or equal to 90 wt %, greater than or equal to 95 wt %, greater than or equal to 98 wt %, greater than or equal to 99 wt %, greater than or equal to 99.5 wt %, or greater than or equal to 99.9 wt %, based on the total weight of the third intermediate product.

Embodiment A10. The process any one of Embodiments A1-A9, further comprising:
a) oxidizing at least a portion of the ethylbenzene in the third intermediate product to form ethylbenzene hydroperoxide, catalytically reacting the ethylbenzene hydroperoxide with propylene to form propylene oxide and 1-phenyl ethanol, and dehydrating the 1-phenyl ethanol to produce styrene monomer;
b) dehydrogenating at least a portion of the ethylbenzene in the third intermediate product to produce styrene monomer; or
c) a combination thereof.

Embodiment A11. The process of process any one of Embodiments A1-A10, further comprising:
a) adding at least a portion of the first residual fraction to the one or more pyrolysis reaction zones as additional feed;
b) sending at least a portion of the residual fraction outside the process for further processing; or
c) a combination thereof.

Embodiment A12. The process of any one of Embodiments A1-A11, wherein the one or more first pyrolysis reaction zones are in series.

Embodiment A13. The process of any one of Embodiments A1-A12, further comprising:
a) removing at least a portion of the first residual fraction from the process;
b) recycling at least a portion of the first residual fraction to the first pyrolysis zone as additional feed;
c) adding at least a portion of the first residual fraction to one or more second pyrolysis zones to produce a fourth intermediate product; or
d) a combination thereof.

Embodiment A14. The process of Embodiment A4, wherein the second pyrolysis conditions and the one or more second pyrolysis reaction zones comprise:
a) thermal pyrolysis conditions in one or more thermal pyrolysis reaction zones;
b) catalytic pyrolysis conditions in one or more catalytic pyrolysis zones; or
c) a combination thereof.

Embodiment A15. The process of Embodiment A14, wherein the thermal pyrolysis conditions comprise:
a) a temperature in the range of from about 200° C. to about 800° C., from 300° C. to 700° C., or from 400° C. to 600° C.;
b) a pressure in the range of from 5 kPa (absolute) to 450 kPa, from 13.3 kPa (absolute) to 400 kPa, from atmospheric pressure to 350 kPa, or from 100 kPa to 300 kPa; or
c) a combination thereof.

Embodiment A16. The process any one of Embodiments A14-A15, wherein the catalytic pyrolysis conditions comprise:
a) a temperature in the range of from 300° C. to 600° C., from 250° C. to 450° C., from 280° C. to 420° C., or from 280° C. to 350° C.;
b) a pressure in the range of from 5 kPa (absolute) to 450 kPa, from 13.3 kPa (absolute) to 400 kPa, from atmospheric pressure to 350 kPa, or from 100 kPa to 300 kPa;
c) a weight hourly space velocity in the range of from 0.1 hr⁻¹ to 100 hr⁻¹, from 0.5 hr⁻¹ to 60 hr⁻¹, from 0.8 hr⁻¹ to 25 hr⁻¹, from 1.0 hr⁻¹ to 25 hr⁻¹, or from 3.0 hr⁻¹ to 25 hr⁻¹; or
d) a combination thereof.

Embodiment A17. The process any one of Embodiments A14-A16, wherein the one or more catalytic pyrolysis zones each independently comprise a medium or large pore zeolite or molecular sieve.

Embodiment A18. The process of Embodiment A17, wherein the medium or large pore zeolite or molecular sieve comprises beta zeolite (BEA structure), ferrierite (FER structure), mordenite (MOR structure), zeolite L (LTL structure), zeolite X (FAU structure), zeolite Y (FAU structure), ZSM-11 (MEL structure), ZSM-22 (TON structure), ZSM-23 (MTT structure), ZSM-5 (MFI structure), or a combination thereof.

Embodiment A19. The process any one of Embodiments A14-A18, wherein the ratio of the styrene content of the first intermediate product to the styrene content of the heavy aromatic waste stream is greater than or equal to 1.01, greater than or equal to 1.02, greater than or equal to 1.05, greater than or equal to 1.10, greater than or equal to 1.15, or greater than or equal to 1.20.

Embodiment A20. The process any one of Embodiments A14-A19, wherein the second intermediate product comprises ethylbenzene in an amount greater than or equal to 30 wt %, greater than or equal to 40 wt %, greater than or equal to 50 wt %, or greater than or equal to 60 wt %, based on the total weight of the second intermediate product.

Embodiment A21. The process any one of Embodiments A14-A20, wherein the third intermediate product comprises ethylbenzene in an amount greater than or equal to 60 wt %, greater than or equal to 70 wt %, greater than or equal to 80 wt %, greater than or equal to 90 wt %, greater than or equal to 95 wt %, greater than or equal to 98 wt %, greater than or equal to 99 wt %, greater than or equal to 99.5 wt %, or greater than or equal to 99.9 wt %, based on the total weight of the third intermediate product.

Embodiment A22. The process any one of Embodiments A14-A21, further comprising:
a) oxidizing at least a portion of the ethylbenzene in the third intermediate product to form ethylbenzene hydroperoxide, catalytically reacting the ethylbenzene hydroperoxide with propylene to form propylene oxide and 1-phenyl ethanol, and dehydrating the 1-phenyl ethanol to produce styrene monomer;
b) dehydrogenating at least a portion of the ethylbenzene in the third intermediate product to produce styrene monomer; or
c) a combination thereof.

Embodiment A23. The process of process any one of Embodiments A14-A22, further comprising:
a) adding at least a portion of the first residual fraction to the one or more pyrolysis reaction zones as additional feed;
b) sending at least a portion of the residual fraction outside the process for further processing; or
c) a combination thereof.

Embodiment A24. The process of any one of Embodiments A14-A23, further comprising subjecting the fourth intermediate product to second separation conditions to produce a fifth intermediate product comprising ethylbenzene and a second residual fraction.

Embodiment A25. The process of any one of Embodiments A14-A24, wherein the one or more second pyrolysis reaction zones are in series.

Embodiment A26. The process of any one of Embodiment A14-A25, further comprising:
a) removing at least a portion of the second residual fraction from the process;
b) recycling at least a portion of the second residual fraction to the first pyrolysis zone as additional feed;
c) adding at least a portion of the second residual fraction to one or more second pyrolysis zones; or
d) a combination thereof.

Embodiment B1. A process to upgrade a heavy aromatic waste stream to chemical feedstocks, the process comprising:
a) subjecting the heavy aromatic waste stream to pyrolysis conditions in one or more first pyrolysis reaction zones to produce a first intermediate product having a higher styrene content than the heavy aromatic waste stream;
b) subjecting the first intermediate product to first separation conditions to produce a second intermediate product and a first residual fraction; and
c) subjecting the second intermediate product to hydrogenation conditions in a hydrogenation reaction to produce a third intermediate product comprising ethylbenzene.

Embodiment B2. The process of Embodiment B1, wherein the first pyrolysis conditions and the one or more first pyrolysis reaction zones comprise:
a) thermal pyrolysis conditions in one or more thermal pyrolysis reaction zones;
b) catalytic pyrolysis conditions in one or more catalytic pyrolysis zones; or
c) a combination thereof.

Embodiment B3. The process of Embodiment B2, wherein the thermal pyrolysis conditions comprise:
a) a temperature in the range of from about 200° C. to about 800° C., from 300° C. to 700° C., or from 400° C. to 600° C.;
b) a pressure in the range of from 5 kPa (absolute) to 450 kPa, from 13.3 kPa (absolute) to 400 kPa, from atmospheric pressure to 350 kPa, or from 100 kPa to 300 kPa; or
c) a combination thereof.

Embodiment B4. The process any one of Embodiments B2-B3, wherein the catalytic pyrolysis conditions comprise:
a) a temperature in the range of from 300° C. to 600° C., from 250° C. to 450° C., from 280° C. to 420° C., or from 280° C. to 350° C.;
b) a pressure in the range of from 5 kPa (absolute) to 450 kPa, from 13.3 kPa (absolute) to 400 kPa, from atmospheric pressure to 350 kPa, or from 100 kPa to 300 kPa;
c) a weight hourly space velocity in the range of from 0.1 hr⁻¹ to 100 hr⁻¹, from 0.5 hr⁻¹ to 60 hr⁻¹, from 0.8 hr⁻¹ to 25 hr⁻¹, from 1.0 hr⁻¹ to 25 hr⁻¹, or from 3.0 hr⁻¹ to 25 hr⁻¹; or
d) a combination thereof.

Embodiment B5. The process any one of Embodiments B2-B4, wherein the one or more catalytic pyrolysis zones each independently comprise a medium or large pore zeolite or molecular sieve.

Embodiment B6. The process of Embodiment B5, wherein the medium or large pore zeolite or molecular sieve comprises beta zeolite (BEA structure), ferrierite (FER structure), mordenite (MOR structure), zeolite L (LTL structure), zeolite X (FAU structure), zeolite Y (FAU structure), ZSM-11 (MEL structure), ZSM-22 (TON structure), ZSM-23 (MTT structure), ZSM-5 (MFI structure), or a combination thereof.

Embodiment B7. The process any one of Embodiments B1-B6, wherein the ratio of the styrene content of the first intermediate product to the styrene content of the heavy aromatic waste stream is greater than or equal to 1.01, greater than or equal to 1.02, greater than or equal to 1.05, greater than or equal to 1.10, greater than or equal to 1.15, or greater than or equal to 1.20.

Embodiment B8. The process any one of Embodiments A1-B7, wherein the second intermediate product comprises ethylbenzene in an amount greater than or equal to 30 wt %, greater than or equal to 40 wt %, greater than or equal to 50 wt %, or greater than or equal to 60 wt %, based on the total weight of the second intermediate product.

Embodiment B9. The process any one of Embodiments B1-B8, wherein the third intermediate product comprises ethylbenzene in an amount greater than or equal to 60 wt %, greater than or equal to 70 wt %, greater than or equal to 80 wt %, greater than or equal to 90 wt %, greater than or equal to 95 wt %, greater than or equal to 98 wt %, greater than or equal to 99 wt %, greater than or equal to 99.5 wt %, or greater than or equal to 99.9 wt %, based on the total weight of the third intermediate product.

Embodiment B10. The process any one of Embodiments B1-B9, further comprising:
a) oxidizing at least a portion of the ethylbenzene in the third intermediate product to form ethylbenzene hydroperoxide, catalytically reacting the ethylbenzene hydroperoxide with propylene to form propylene oxide and 1-phenyl ethanol, and dehydrating the 1-phenyl ethanol to produce styrene monomer;
b) dehydrogenating at least a portion of the ethylbenzene in the third intermediate product to produce styrene monomer; or
c) a combination thereof.

Embodiment B11. The process of process any one of Embodiments B1-B10, further comprising:
a) adding at least a portion of the first residual fraction to the one or more pyrolysis reaction zones as additional feed;
b) sending at least a portion of the residual fraction outside the process for further processing; or
c) a combination thereof.

Embodiment B12. The process of any one of Embodiments B1-B11, wherein the one or more first pyrolysis reaction zones are in series.

Embodiment B13. The process of any one of Embodiments B1-B12, further comprising:
a) removing at least a portion of the first residual fraction from the process;
b) recycling at least a portion of the first residual fraction to the first pyrolysis zone as additional feed;
c) adding at least a portion of the first residual fraction to one or more second pyrolysis zones to produce a fourth intermediate product; or
d) a combination thereof.

Embodiment B14. The process of Embodiment B13, wherein the second pyrolysis conditions and the one or more second pyrolysis reaction zones comprise:
a) thermal pyrolysis conditions in one or more thermal pyrolysis reaction zones;
b) catalytic pyrolysis conditions in one or more catalytic pyrolysis zones; or
c) a combination thereof.

Embodiment B15. The process of Embodiment B14, wherein the thermal pyrolysis conditions comprise:
a) a temperature in the range of from about 200° C. to about 800° C., from 300° C. to 700° C., or from 400° C. to 600° C.;
b) a pressure in the range of from 5 kPa (absolute) to 450 kPa, from 13.3 kPa (absolute) to 400 kPa, from atmospheric pressure to 350 kPa, or from 100 kPa to 300 kPa; or
c) a combination thereof.

Embodiment B16. The process any one of Embodiments B14-B15, wherein the catalytic pyrolysis conditions comprise:
a) a temperature in the range of from 300° C. to 600° C., from 250° C. to 450° C., from 280° C. to 420° C., or from 280° C. to 350° C.;
b) a pressure in the range of from 5 kPa (absolute) to 450 kPa, from 13.3 kPa (absolute) to 400 kPa, from atmospheric pressure to 350 kPa, or from 100 kPa to 300 kPa;
c) a weight hourly space velocity in the range of from 0.1 hr⁻¹ to 100 hr⁻¹, from 0.5 hr⁻¹ to 60 hr⁻¹, from 0.8 hr⁻¹ to 25 hr⁻¹, from 1.0 hr⁻¹ to 25 hr⁻¹, or from 3.0 hr⁻¹ to 25 hr⁻¹; or
d) a combination thereof.

Embodiment B17. The process any one of Embodiments B14-B16, wherein the one or more catalytic pyrolysis zones each independently comprise a medium or large pore zeolite or molecular sieve.

Embodiment B18. The process of Embodiment B17, wherein the medium or large pore zeolite or molecular sieve comprises beta zeolite (BEA structure), ferrierite (FER structure), mordenite (MOR structure), zeolite L (LTL structure), zeolite X (FAU structure), zeolite Y (FAU structure), ZSM-11 (MEL structure), ZSM-22 (TON structure), ZSM-23 (MTT structure), ZSM-5 (MFI structure), or a combination thereof.

Embodiment B19. The process any one of Embodiments B14-B18, wherein the ratio of the styrene content of the first intermediate product to the styrene content of the heavy aromatic waste stream is greater than or equal to 1.01, greater than or equal to 1.02, greater than or equal to 1.05, greater than or equal to 1.10, greater than or equal to 1.15, or greater than or equal to 1.20.

Embodiment B20. The process any one of Embodiments B14-B19, wherein the second intermediate product comprises ethylbenzene in an amount greater than or equal to 30 wt %, greater than or equal to 40 wt %, greater than or equal to 50 wt %, or greater than or equal to 60 wt %, based on the total weight of the second intermediate product.

Embodiment B21. The process any one of Embodiments B14-B20, wherein the third intermediate product comprises ethylbenzene in an amount greater than or equal to 60 wt %, greater than or equal to 70 wt %, greater than or equal to 80 wt %, greater than or equal to 90 wt %, greater than or equal to 95 wt %, greater than or equal to 98 wt %, greater than or equal to 99 wt %, greater than or equal to 99.5 wt %, or greater than or equal to 99.9 wt %, based on the total weight of the third intermediate product.

Embodiment B22. The process any one of Embodiments B14-B21, further comprising:
a) oxidizing at least a portion of the ethylbenzene in the third intermediate product to form ethylbenzene hydroperoxide, catalytically reacting the ethylbenzene hydroperoxide with propylene to form propylene oxide and 1-phenyl ethanol, and dehydrating the 1-phenyl ethanol to produce styrene monomer;
b) dehydrogenating at least a portion of the ethylbenzene in the third intermediate product to produce styrene monomer; or
c) a combination thereof.

Embodiment B23. The process of process any one of Embodiments B14-B22, further comprising:
a) adding at least a portion of the first residual fraction to the one or more pyrolysis reaction zones as additional feed;
b) sending at least a portion of the residual fraction outside the process for further processing; or
c) a combination thereof.

Embodiment B24. The process of any one of Embodiments B14-B23, further comprising subjecting the fourth intermediate product to second separation conditions to produce a fifth intermediate product comprising ethylbenzene and a second residual fraction.

Embodiment B25. The process of any one of Embodiments B14-B24, wherein the one or more second pyrolysis reaction zones are in series.

Embodiment B26. The process of any one of Embodiment B14-B25, further comprising:
a) removing at least a portion of the second residual fraction from the process;
b) recycling at least a portion of the second residual fraction to the first pyrolysis zone as additional feed;
c) adding at least a portion of the second residual fraction to one or more second pyrolysis zones; or
d) a combination thereof.

Embodiment C1. A process to upgrade a heavy aromatic waste stream to chemical feedstocks, the process comprising:
a) subjecting the heavy aromatic waste stream to pyrolysis conditions in one or more first pyrolysis reaction zones to produce a first intermediate product having a higher styrene content than the heavy aromatic waste stream;
b) subjecting the first intermediate product to first separation conditions to produce a second intermediate product and a first residual fraction;
c) subjecting the second intermediate product to hydrogenation conditions in a hydrogenation reaction to produce a third intermediate product comprising ethylbenzene; and
d) subjecting the third intermediate product to second separation conditions to produce a fourth intermediate product and a second residual fraction.

Embodiment C2. The process of Embodiment C1, wherein the first pyrolysis conditions and the one or more first pyrolysis reaction zones comprise:
a) thermal pyrolysis conditions in one or more thermal pyrolysis reaction zones;
b) catalytic pyrolysis conditions in one or more catalytic pyrolysis zones; or
c) a combination thereof.

Embodiment C3. The process of Embodiment C2, wherein the thermal pyrolysis conditions comprise:
a) a temperature in the range of from about 200° C. to about 800° C., from 300° C. to 700° C., or from 400° C. to 600° C.;
b) a pressure in the range of from 5 kPa (absolute) to 450 kPa, from 13.3 kPa (absolute) to 400 kPa, from atmospheric pressure to 350 kPa, or from 100 kPa to 300 kPa; or
c) a combination thereof.

Embodiment C4. The process any one of Embodiments C2-C3, wherein the catalytic pyrolysis conditions comprise:
a) a temperature in the range of from 300° C. to 600° C., from 250° C. to 450° C., from 280° C. to 420° C., or from 280° C. to 350° C.;
b) a pressure in the range of from 5 kPa (absolute) to 450 kPa, from 13.3 kPa (absolute) to 400 kPa, from atmospheric pressure to 350 kPa, or from 100 kPa to 300 kPa;
c) a weight hourly space velocity in the range of from 0.1 hr⁻¹ to 100 hr⁻¹, from 0.5 hr⁻¹ to 60 hr⁻¹, from 0.8 hr⁻¹ to 25 hr⁻¹, from 1.0 hr⁻¹ to 25 hr⁻¹, or from 3.0 hr⁻¹ to 25 hr⁻¹; or
d) a combination thereof.

Embodiment C5. The process any one of Embodiments C2-C4, wherein the one or more catalytic pyrolysis zones each independently comprise a medium or large pore zeolite or molecular sieve.

Embodiment C6. The process of Embodiment C5, wherein the medium or large pore zeolite or molecular sieve comprises beta zeolite (BEA structure), ferrierite (FER structure), mordenite (MOR structure), zeolite L (LTL structure), zeolite X (FAU structure), zeolite Y (FAU structure), ZSM-11 (MEL structure), ZSM-22 (TON structure), ZSM-23 (MTT structure), ZSM-5 (MFI structure), or a combination thereof.

Embodiment C7. The process any one of Embodiments C1-C6, wherein the ratio of the styrene content of the first intermediate product to the styrene content of the heavy aromatic waste stream is greater than or equal to 1.01, greater than or equal to 1.02, greater than or equal to 1.05, greater than or equal to 1.10, greater than or equal to 1.15, or greater than or equal to 1.20.

Embodiment C8. The process any one of Embodiments C1-C7, wherein the second intermediate product comprises ethylbenzene in an amount greater than or equal to 30 wt %, greater than or equal to 40 wt %, greater than or equal to 50 wt %, or greater than or equal to 60 wt %, based on the total weight of the second intermediate product.

Embodiment C9. The process any one of Embodiments C1-C8, wherein the fourth intermediate product comprises ethylbenzene in an amount greater than or equal to 60 wt %, greater than or equal to 70 wt %, greater than or equal to 80 wt %, greater than or equal to 90 wt %, greater than or equal to 95 wt %, greater than or equal to 98 wt %, greater than or equal to 99 wt %, greater than or equal to 99.5 wt %, or greater than or equal to 99.9 wt %, based on the total weight of the fourth intermediate product.

Embodiment C10. The process any one of Embodiments C1-C9, further comprising:
a) oxidizing at least a portion of the ethylbenzene in the fourth intermediate product to form ethylbenzene hydroperoxide, catalytically reacting the ethylbenzene hydroperoxide with propylene to form propylene oxide and 1-phenyl ethanol, and dehydrating the 1-phenyl ethanol to produce styrene monomer;
b) dehydrogenating at least a portion of the ethylbenzene in the fourth intermediate product to produce styrene monomer; or
c) a combination thereof.

Embodiment C11. The process of process any one of Embodiments C1-C10, further comprising:
a) adding at least a portion of the first residual fraction to the one or more pyrolysis reaction zones as additional feed;
b) sending at least a portion of the residual fraction outside the process for further processing; or
c) a combination thereof.

Embodiment C12. The process of any one of Embodiments C1-C11, wherein the one or more first pyrolysis reaction zones are in series.

Embodiment C13. The process of any one of Embodiments C1-C12, further comprising:
a) removing at least a portion of the first residual fraction and/or the second residual fraction from the process;
b) recycling at least a portion of the first residual fraction and/or the second residual fraction to the first pyrolysis zone as additional feed;
c) subjecting at least a portion of the first residual fraction and/or the second residual fraction to second pyrolysis conditions in one or more second pyrolysis zones to produce a fifth intermediate product; or
d) a combination thereof.

Embodiment C14. The process of Embodiment C13, wherein the second pyrolysis conditions and the one or more second pyrolysis reaction zones comprise:
a) thermal pyrolysis conditions in one or more thermal pyrolysis reaction zones;
b) catalytic pyrolysis conditions in one or more catalytic pyrolysis zones; or
c) a combination thereof.

Embodiment C15. The process of Embodiment C14, wherein the thermal pyrolysis conditions comprise:
a) a temperature in the range of from about 200° C. to about 800° C., from 300° C. to 700° C., or from 400° C. to 600° C.;
b) a pressure in the range of from 5 kPa (absolute) to 450 kPa, from 13.3 kPa (absolute) to 400 kPa, from atmospheric pressure to 350 kPa, or from 100 kPa to 300 kPa; or
c) a combination thereof.

Embodiment C16. The process any one of Embodiments C14-C15, wherein the catalytic pyrolysis conditions comprise:
a) a temperature in the range of from 300° C. to 600° C., from 250° C. to 450° C., from 280° C. to 420° C., or from 280° C. to 350° C.;
b) a pressure in the range of from 5 kPa (absolute) to 450 kPa, from 13.3 kPa (absolute) to 400 kPa, from atmospheric pressure to 350 kPa, or from 100 kPa to 300 kPa;
c) a weight hourly space velocity in the range of from 0.1 hr⁻¹ to 100 hr⁻¹, from 0.5 hr⁻¹ to 60 hr⁻¹, from 0.8 hr⁻¹ to 25 hr⁻¹, from 1.0 hr⁻¹ to 25 hr⁻¹, or from 3.0 hr⁻¹ to 25 hr⁻¹; or
d) a combination thereof.

Embodiment C17. The process any one of Embodiments C14-C16, wherein the one or more catalytic pyrolysis zones each independently comprise a medium or large pore zeolite or molecular sieve.

Embodiment C18. The process of Embodiment C17, wherein the medium or large pore zeolite or molecular sieve comprises beta zeolite (BEA structure), ferrierite (FER structure), mordenite (MOR structure), zeolite L (LTL structure), zeolite X (FAU structure), zeolite Y (FAU structure), ZSM-11 (MEL structure), ZSM-22 (TON structure), ZSM-23 (MTT structure), ZSM-5 (MFI structure), or a combination thereof.

Embodiment C19. The process any one of Embodiments C14-C18, wherein the ratio of the styrene content of the first intermediate product to the styrene content of the heavy aromatic waste stream is greater than or equal to 1.01, greater than or equal to 1.02, greater than or equal to 1.05, greater than or equal to 1.10, greater than or equal to 1.15, or greater than or equal to 1.20.

Embodiment C20. The process any one of Embodiments C14-C19, wherein the second intermediate product comprises ethylbenzene in an amount greater than or equal to 30 wt %, greater than or equal to 40 wt %, greater than or equal to 50 wt %, or greater than or equal to 60 wt %, based on the total weight of the second intermediate product.

Embodiment C21. The process any one of Embodiments C14-C20, wherein the fourth intermediate product comprises ethylbenzene in an amount greater than or equal to 60 wt %, greater than or equal to 70 wt %, greater than or equal to 80 wt %, greater than or equal to 90 wt %, greater than or equal to 95 wt %, greater than or equal to 98 wt %, greater than or equal to 99 wt %, greater than or equal to 99.5 wt %, or greater than or equal to 99.9 wt %, based on the total weight of the fourth intermediate product.

Embodiment C22. The process any one of Embodiments C14-C21, further comprising:
a) oxidizing at least a portion of the ethylbenzene in the fourth intermediate product to form ethylbenzene hydroperoxide, catalytically reacting the ethylbenzene hydroperoxide with propylene to form propylene oxide and 1-phenyl ethanol, and dehydrating the 1-phenyl ethanol to produce styrene monomer;
b) dehydrogenating at least a portion of the ethylbenzene in the fourth intermediate product to produce styrene monomer; or
c) a combination thereof.

Embodiment C23. The process of process any one of Embodiments C14-C22, further comprising:
a) adding at least a portion of the first residual fraction to the one or more pyrolysis reaction zones as additional feed;
b) sending at least a portion of the residual fraction outside the process for further processing; or
c) a combination thereof.

Embodiment C24. The process of any one of Embodiments C14-C23, further comprising subjecting the fifth intermediate product to third separation conditions to produce a sixth intermediate product comprising ethylbenzene and a third residual fraction.

Embodiment C25. The process of any one of Embodiments C14-C24, wherein the one or more second pyrolysis reaction zones are in series.

Embodiment C26. The process of any one of Embodiment C14-C25, further comprising:
a) removing at least a portion of the third residual fraction from the process;
b) recycling at least a portion of the third residual fraction to the first pyrolysis zone as additional feed;
c) adding at least a portion of the third residual fraction to one or more second pyrolysis zones; or
d) a combination thereof.

Embodiment D1. A method for converting a fuel oil to propylene comprising: (a) providing a fuel oil comprising a plurality of aromatic compounds comprising: (i) from about 1.0 wt % to about 2.0 wt % ethylbenzene; and (ii) from about 1.0 wt % to about 2.0 wt % styrene, wherein the fuel oil comprises no more than 1.0 wt % benzene and 0.2 wt % propylene; and (b) catalytically cracking the fuel oil in the presence of a catalyst to produce a cracked product comprising: (iii) from about 10 wt % to about 19 wt % propylene; and (iv) from about 36 wt % to about 50 wt % benzene.

Embodiment D2. The method of Embodiment D1, wherein the method is further characterized by one or more of the following additional elements:
a) Element 1: catalytic cracking the fuel oil occurs at a temperature greater than about 380° C.;
b) Element 2: catalytic cracking the fuel oil occurs at a temperature greater than about 480° C.;
c) Element 3: catalytic cracking the fuel oil occurs at a temperature greater than about 580° C.;
d) Element 4: the catalyst is a zeolite; and
e) Element 5: the catalyst is a H-ZSM-5 zeolite.

Embodiment E1. A method for converting a fuel oil to propylene comprising:
a) providing a fuel oil comprising a plurality of aromatic compounds comprising:
   i) from about 1.0 wt % to about 2.0 wt % ethylbenzene; and
   ii) from about 1.0 wt % to about 2.0 wt % styrene;
   wherein the fuel oil comprises no more than 1.0 wt % benzene and 0.2 wt % propylene; and
b) catalytically cracking the fuel oil in the presence of a catalyst to produce a cracked product comprising:
   i) from about 10 wt % to about 19 wt % propylene; and
   ii) from about 36 wt % to about 50 wt % benzene.

Embodiment E2. The method of Embodiment E1, wherein catalytic cracking the fuel oil occurs at a temperature greater than about 380° C., greater than about 480° C., or greater than about 580° C.

Embodiment E3. The method of Embodiment E1 or E2, wherein the catalyst is a zeolite.

Embodiment E4. The method of Embodiment E3, wherein the zeolite is a H-ZSM-5 zeolite.

The presently disclosed processes are exemplified in the examples below. These examples are included to demonstrate embodiments of the appended claims. However, these are exemplary only, and the invention can be broadly applied to embodiments not demonstrated in the examples. Those of skill in the art should appreciate that many changes can be made in the specific embodiments which are disclosed and still obtain a like or similar result without departing from the spirit and scope of the disclosure herein. In no way should the following examples be read to limit, or to define, the scope of the appended claims.

### EXAMPLES

The following examples are included to demonstrate embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### BACKGROUND

Characterization of liquid products: The liquid products from the two traps were characterized by Gas Chromatography (GC) and proton NMR (1H NMR).

The GC analysis of the liquid product for each run was performed using an Agilent 7890 GC (Agilent Technologies, Santa Clara, CA) equipped with a standard non-polar column and a flame ionization detector. For the GC data, the weight percent for x<nC7 (having boiling point <98° C. named LF1), nC7<x<nC11 (having boiling point 98° C. <BP<203° C. named LF2), nC12<x<nC28 (having boiling point 203° C.<BP<434° C. named LF3), x>C28 (having boiling point >434° C. named LF4) were used to characterize the liquid product.

NMR data were used to characterize the percent of aromatic protons, paraffinic protons and olefinic protons in the liquid product. The examples were analyzed with an addition of CDCl3 (0.6 g of depolymerize polymer/metal oxide mixture with 0.4 g of CDCl3). The data were collected on a Bruker AV500 MHz NMR spectrometer (Bruker Corporation, Billerica, MA) at 25° C. with a 5 mm Prodigy probe. One dimension 1H NMR data were processed using TOPSPIN® software (Bruker) with an exponential line broadening window function. Quantitative measurements were performed with a 15 second relaxation delay, a 30° flip angle pulse, and 32 scans to facilitate accurate integrals. The spectral integrations for aromatic olefinic, and paraffinic protons were obtained and used to quantify relative ratios of these protons.

### FTIR Process

FTIR spectroscopy was carried out as follows. An iS50 FTIR spectrometer equipped with a DTGS detector from Thermo Scientific was used for FTIR spectral acquisition. The sample compartment was fitted with an attenuated total reflectance (ATR) accessory. The ATR cell was obtained from Pike Technologies and was equipped with a 3-bounce zinc selenide (ZnSe) crystal. A sample volume of about 0.05 mLs was required for analysis. Those familiar with FTIR spectroscopy will recognize that when multiple spectra are overlaid in the same plot, these spectra can be shown with similar baselines or offset baselines. This choice is purely for optimal viewing of changes occurring and does not impact peak height or peak absorbance measurements as such measurements are made from the baseline to the top of the peak.

### Thermal and Catalytic Cracking Studies

In each test, approximately 0.5-1 mg of a waste feed material and ˜50 mg of catalyst was added to a Frontier Lab Tandem Micro-Reactor system attached to an Agilent GC/MS with a flow of 200 ml/min of helium through the headspace above the polymer recyclate. Each sample was heated to 500 or 550° C. for 1 minute. The pyrolysate gas passed through a quartz tube containing an optional catalyst maintained at a specified cracking temperature. Reaction product gas was cryo-trapped at the head of the GC/MS column for subsequent separation and identification.

### Determination of Catalytic Pyrolysis Reaction Product

The composition of the reaction product was measured with Pyrolysis Gas Chromatography-Mass Spectrometry (Pyrolysis-GC/MS-FID) analysis using a Rx-3050TR Tandem micro-Reactor, Frontier labs, Fukushima, JP; and an Agilent 8890 GC equipped with a flame ionization detector (FID) and 5977B MSD and UA1 (30 m×250 μm×2 μm) GC column, Agilent Technologies, Santa Clara, California. The GC is also equipped with a splitter to split the column effluent between the FID and MSD.

GC/MS, or gas chromatography-mass spectrometry, was used to identify the composition of the reaction product stream. Of particular interest are the ethane and ethylene (C₂), propane and propylene (C₃), mixed butanes and butenes (C₄), and benzene/toluene/xylenes (BTX). The quantification was done using the FID signal from each experiment.

### Thermal Pyrolysis

Table 1 shows results of non-catalytic pyrolysis tests on residual fuel oil and polystyrene pyrolysis oil heavies. Results show similar increases in styrene and ethylbenzene after thermal pyrolysis at 500° C.

TABLE 1

| | | Polystyrene |
|---|---|---|
| | Residual Fuel | Pyrolysis Oil |
| Product | Oil | Heavies |
| Styrene (wt %) | 67.5 | 79.3 |
| Toluene (wt %) | 4.6 | 7.5 |
| A-Me-Styrene (wt %) | 2.7 | 4.6 |
| Ethylbenzene (wt %) | 23.5 | 1.7 |
| Benzeneacetaldehyde (wt %) | <0.1 | 1.8 |
| Acetophenone (wt %) | <0.1 | 1.8 |
| 2-Phenelpropenal (wt %) | <0.1 | 1.6 |

### Catalytic Pyrolysis

Table 2 shows results of catalytic pyrolysis tests on residual fuel oil and polystyrene pyrolysis oil heavies. Results show similar increases in styrene and ethylbenzene after catalytic pyrolysis using mordenite at 500° C.

TABLE 2

| | | Polystyrene |
|---|---|---|
| | Residual Fuel | Pyrolysis Oil |
| Product | Oil | Heavies |
| Benzene (wt %) | 60.1 | 62.6 |
| Naphthalene (wt %) | 16.2 | 18.3 |
| C₂^{═} and C₃^{═} (wt %) | 4.8 | 7.1 |
| Toluene (wt %) | 7.9 | 6.0 |
| Me-Naphthalene (wt %) | 3.2 | 2.6 |

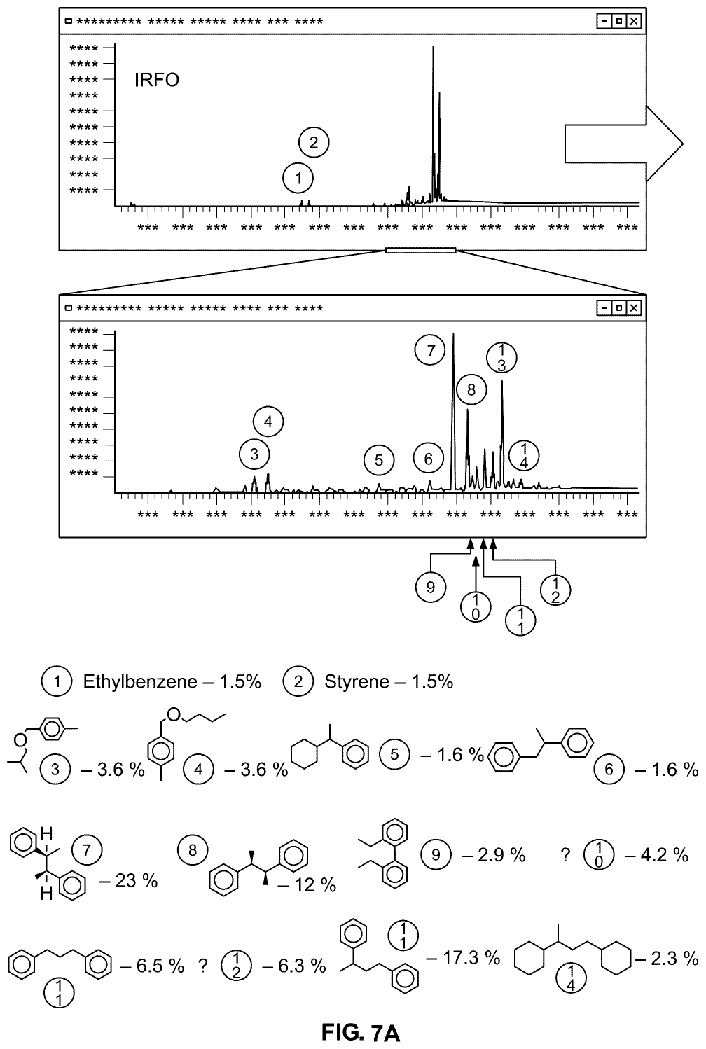

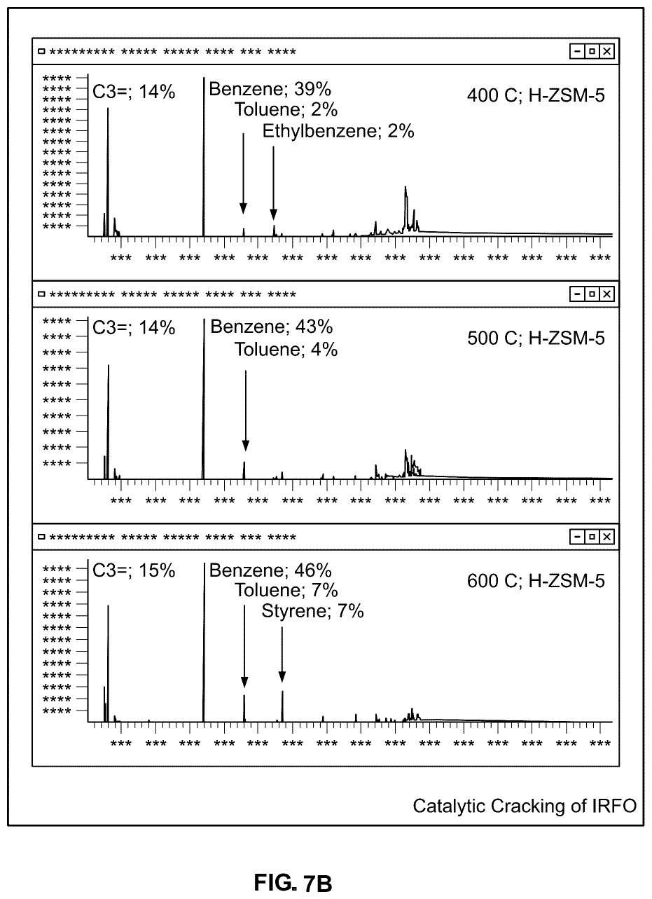

FIG. 7A presents two spectra produced from the fuel oil used in the below Examples. The lower of the two spectra presents a close up view of a portion of the upper spectra. Various peaks are numbered corresponding to different chemical species identified below the spectra. The percentages represent the wt % of the chemical species present in the fuel oil. For example, 1.5 wt % ethylbenzene. FIG. 7B presents three spectra of the product produced by catalytically cracking the fuel oil identified in the spectra of FIG. 7A at three different temperatures: 400° C., 500° C., and 600° C.

### Example 1

A fuel oil having the composition indicated by the spectra shown in FIG. 7A was catalytically cracked at 400° C. in the presence of a H-ZSM-5 catalyst in a Frontier Lab tandem microreactor GC/MS system to produce a cracked product comprising about 14 wt % propylene, about 39 wt % benzene, about 2 wt % toluene, and about 2 wt % ethylbenzene as shown in FIG. 7B.

### Example 2

A fuel oil having the composition indicated by the spectra shown in FIG. 7A was catalytically cracked at 500° C. in the presence of a H-ZSM-5 catalyst in a Frontier Lab tandem microreactor GC/MS system to produce a cracked product comprising about 14 wt % propylene, about 43 wt % benzene, and about 4 wt % toluene as shown in FIG. 7B.

### Example 3

A fuel oil having the composition indicated by the spectra shown in FIG. 7A was catalytically cracked at 600° C. in the presence of a H-ZSM-5 catalyst in a Frontier Lab tandem microreactor GC/MS system to produce a cracked product comprising about 15 wt % propylene, about 46 wt % benzene, about 7 wt % toluene, and about 7 wt % styrene as shown in FIG. 7B.

### Hydrogenation

Table 3 shows effectiveness of hydrogenating pyrolysis product of polystyrene pyrolysis heavies. 1H NMR measurements of a sample of pyrolysis product of polystyrene are shown before and after hydrogenation using NiMo—S(KL-8234) catalyst. Results show a reduction in olefinic and aromatic double bonds after hydrogenation.

TABLE 3

| | | | | Styrene | | |
|---|---|---|---|---|---|---|
| | | | | double | | |
| Sample | Saturated | Oxygenates | Olefinic | bond | Aromatic | Aldehyde |
| Starting | 62.002 | 2.159 | 2.7475 | 1.5939 | 31.3861 | 0.1114 |
| Material | | | | | | |
| Hydrogenated | 71.315 | 0.9257 | 0.463 | 0.1905 | 27.1028 | 0.003 |
| Material | | | | | | |

Table 4 shows the yield of the product composition resulting from hydrogenation of a liquid feed comprising 10 wt % pyrolysis oil made from the thermal pyrolysis of polystyrene and 90 wt % decalin used as a diluent. A stainless steel continuous packed bed reactor loaded with 3 grams of a sulfided NiMo catalyst (NiMo—S) was used to hydrogenate the liquid feed flowing through the catalyst bed at a flow rate of 0.025 mL/min with a hydrogen (H2) gas flow rate of 100 sccm. Results show complete conversion of styrene with greater than 98% of the styrene present in the feed being converted to ethylbenzene. The GC analysis of the product composition was performed using an Agilent 7890 GC (Agilent Technologies, Santa Clara, CA) equipped with a standard non-polar column and a flame ionization detector. For the GC data, the weight percent for x<nC7 (having boiling point <98° C. named LF1), nC7<x<nC11 (having boiling point 98° C.<BP<203° C. named LF2), nC12<x<nC28 (having boiling point 203° C.<BP<434° C. named LF3), x>C28 (having boiling point >434° C. named LF4) were used to characterize the product.

TABLE 4

| Component | Ethylbenzene | Ethylcyclohexane | Styrene | Heavies |
|---|---|---|---|---|
| Yield (mol %) | 60% | 1% | 0% | 39% |

For the sake of brevity, only certain ranges are explicitly disclosed herein. However, in addition to recited ranges, any lower limit may be combined with any upper limit to recite a range not explicitly recited, as well as, ranges from any lower limit may be combined with any other lower limit to recite a range not explicitly recited, in the same way, ranges from any upper limit may be combined with any other upper limit to recite a range not explicitly recited. Additionally, within a range includes every point or individual value between its end points even though not explicitly recited. Thus, every point or individual value may serve as its own lower or upper limit combined with any other point or individual value or any other lower or upper limit, to recite a range not explicitly recited.

Although the present invention and its advantages have been described in detail, it should be understood that various changes, substitutions and alterations can be made herein without departing from the spirit and scope of the invention as defined by the appended claims. Moreover, the scope of the present application is not intended to be limited to the particular embodiments of the processes, machines, means, processes, and/or steps described in the specification. As one of the ordinary skill in the art will readily appreciate from the disclosure of the present invention, processes, machines, means, processes, and/or steps, presently existing or later to be developed that perform substantially the same function or achieve substantially the same result as the corresponding embodiments described herein, may be utilized according to the present invention. Accordingly, the appended claims are intended to include within their scope such processes, machines, means, processes, and/or steps.

What is claimed is:

1. A process to upgrade a heavy aromatic waste stream to chemical feedstocks, the process comprising:
a) subjecting the heavy aromatic waste stream to first pyrolysis conditions in one or more first pyrolysis reaction zones to produce a first intermediate product having a higher styrene content than the heavy aromatic waste stream;
b) subjecting the first intermediate product to hydrogenation conditions in a hydrogenation reaction zone to produce a second intermediate product comprising ethylbenzene; and
c) subjecting the second intermediate product to separation conditions to produce a third intermediate product comprising ethylbenzene and a first residual fraction.

2. The process of claim 1, wherein the first pyrolysis conditions and the one or more first pyrolysis reaction zones comprise:
a) thermal pyrolysis conditions in one or more thermal pyrolysis reaction zones;
b) catalytic pyrolysis conditions in one or more catalytic pyrolysis zones; or
c) a combination thereof.

3. The process of claim 1, wherein the one or more first pyrolysis reaction zones are in series.

4. The process of claim 1, further comprising:
a) removing at least a portion of the first residual fraction from the process;
b) recycling at least a portion of the first residual fraction to the one or more first pyrolysis zones as additional feed;
c) subjecting at least a portion of the first residual fraction to second pyrolysis conditions in one or more second pyrolysis zones to produce a fourth intermediate product; or
d) a combination thereof.

5. The process of claim 4, wherein the second pyrolysis conditions and the one or more second pyrolysis reaction zones comprise:
a) thermal pyrolysis conditions in one or more thermal pyrolysis reaction zones;
b) catalytic pyrolysis conditions in one or more catalytic pyrolysis zones; or
c) a combination thereof.

6. The process of claim 4, further comprising subjecting the fourth intermediate product to second separation conditions to produce a fifth intermediate product comprising ethylbenzene and a second residual fraction.

7. The process of claim 6, further comprising:
a) removing at least a portion of the second residual fraction from the process;
b) recycling at least a portion of the second residual fraction to the first pyrolysis zone as additional feed;
c) adding at least a portion of the second residual fraction to one or more second pyrolysis zones; or
d) a combination thereof.

8. A process to upgrade a heavy aromatic waste stream to chemical feedstocks, the process comprising:
a) subjecting the heavy aromatic waste stream to first pyrolysis conditions in one or more first pyrolysis reaction zones to produce a first intermediate product having a higher styrene content than the heavy aromatic waste stream;
b) subjecting the first intermediate product to separation conditions to produce a second intermediate product and a first residual fraction; and
c) subjecting the second intermediate product to hydrogenation conditions in a hydrogenation reaction to produce a third intermediate product comprising ethylbenzene.

9. The process of claim 8, wherein the first pyrolysis conditions and the one or more first pyrolysis reaction zones comprise:
a) thermal pyrolysis conditions in one or more thermal pyrolysis reaction zones;
b) catalytic pyrolysis conditions in one or more catalytic pyrolysis zones; or
c) a combination thereof.

10. The process of claim 8, wherein the one or more first pyrolysis reaction zones are in series.

11. The process of claim 8, further comprising:
a) removing at least a portion of the first residual fraction from the process;
b) recycling at least a portion of the first residual fraction to the first pyrolysis zone as additional feed;
c) subjecting at least a portion of the first residual fraction to second pyrolysis conditions in one or more second pyrolysis zones to produce a fourth intermediate product; or
d) a combination thereof.

12. The process of claim 11, wherein the pyrolysis conditions and the one or more second pyrolysis reaction zones comprise:
a) thermal pyrolysis conditions in one or more thermal pyrolysis reaction zones;
b) catalytic pyrolysis conditions in one or more catalytic pyrolysis zones; or
c) a combination thereof.

13. The process of claim 10, further comprising subjecting the fourth intermediate product to separation conditions to produce a fifth intermediate product comprising ethylbenzene and a second residual fraction.

14. The process of claim 13, further comprising:
a) removing at least a portion of the second residual fraction from the process;
b) recycling at least a portion of the second residual fraction to the first pyrolysis zone as additional feed;
c) adding at least a portion of the second residual fraction to one or more second pyrolysis zones; or
d) a combination thereof.

15. A process to upgrade a heavy aromatic waste stream to chemical feedstocks, the process comprising:
a) subjecting the heavy aromatic waste stream to pyrolysis conditions in one or more first pyrolysis reaction zones to produce a first intermediate product having a higher styrene content than the heavy aromatic waste stream;
b) subjecting the first intermediate product to first separation conditions to produce a second intermediate product and a first residual fraction;
c) subjecting the second intermediate product to hydrogenation conditions in a hydrogenation reaction to produce a third intermediate product comprising ethylbenzene; and
d) subjecting the third intermediate product to second separation conditions to produce a fourth intermediate product and a second residual fraction.

16. The process of claim 15, wherein the pyrolysis conditions and the one or more first pyrolysis reaction zones comprise:
a) thermal pyrolysis conditions in one or more thermal pyrolysis reaction zones;
b) catalytic pyrolysis conditions in one or more catalytic pyrolysis zones; or
c) a combination thereof.

17. The process of claim 15, further comprising:
a) removing at least a portion of the first residual fraction and/or the second residual fraction from the process;
b) recycling at least a portion of the first residual fraction and/or the second residual fraction to the first pyrolysis zone as additional feed;
c) subjecting at least a portion of the first residual fraction and/or the second residual fraction to second pyrolysis conditions in one or more second pyrolysis zones to produce a fifth intermediate product; or
d) a combination thereof.

18. The process of claim 17, wherein the pyrolysis conditions and the one or more second pyrolysis reaction zones comprise:
a) thermal pyrolysis conditions in one or more thermal pyrolysis reaction zones;
b) catalytic pyrolysis conditions in one or more catalytic pyrolysis zones; or
c) a combination thereof.

19. The process of claim 15, further comprising subjecting the fifth intermediate product to third separation conditions to produce a sixth intermediate product comprising ethylbenzene and a third residual fraction.

20. The process of claim 19, further comprising:
a) removing at least a portion of the third residual fraction from the process;
b) recycling at least a portion of the third residual fraction to the first pyrolysis zone as additional feed;
c) adding at least a portion of the third residual fraction to one or more second pyrolysis zones; or
d) a combination thereof.
